(19) United States
(12) Patent Application Publication
(10) Pub. No.: US 2023/0257823 A1
(43) Pub. Date: Aug. 17, 2023

(54) BIOMARKERS FOR IDENTIFYING PATIENTS AT HIGH RISK OF PROGRESSING FROM BARRETT'S ESOPHAGUS TO ESOPHAGEAL ADENOCARCINOMA

(71) Applicant: Rutgers, The State University of New Jersey, New Brunswick, NJ (US)

(72) Inventor: Zhongren Zhou, New Brunswick, NJ (US)

(73) Assignee: Rutgers, The State University of New Jersey, New Brunswick, NJ (US)

(21) Appl. No.: 18/005,472

(22) PCT Filed: Jul. 14, 2021
(86) PCT No.: PCT/US2021/041638

§ 371 Date: Jan. 13, 2023

Related U.S. Application Data
(60) Provisional application No. 63/052,050, filed on Jul. 15, 2020.

(51) Int. Cl. C12Q 1/6886; C12Q 1/6858

(52) U.S. Cl. CPC C12Q 1/6886; C12Q 1/6858; C12Q 2600/154; C12Q 2600/118; C12Q 1/6806

(57) ABSTRACT

The identification of hypermethylated gene loci associated with a high risk of progressing from Barrett's esophagus to esophageal adenocarcinoma is described. The genes with hypermethylated loci include one or more of KLHL14, USP44, TMEM178, TRIM71, CTNNA2, NCAM1, CPXM1, SNCB, BMP3, SHISA3, BDNF, COL2A1, CBS, CLEC4GP1, LRAT, TTYH1, TMEM90B, NTN1, VASH2 and LBXCOR1. The hypermethylated DNA loci can be used as biomarkers to guide diagnostic and treatment decisions for subjects with Barrett's esophagus.

### CROSS REFERENCE TO RELATED APPLICATIONS

This application claims the benefit of U.S. Provisional Application No. 63/052,050, filed Jul. 15, 2020, which is herein incorporated by reference in its entirety.

### FIELD

This disclosure concerns identification of hypermethylated gene loci that can be used to identify Barrett's esophagus patients at high risk of developing esophageal adenocarcinoma (EAC).

### BACKGROUND

The incidence of esophageal adenocarcinoma (EAC) has increased 600-700% in North America since the 1980s. The overall 5-year survival rate for EAC is only 18-22% because most patients present at an advanced stage of disease, such as when they experience discomfort on swallowing. The single major risk factor for the development of EAC is Barrett's esophagus (BE), which is characterized by replacement of the normal squamous mucosa of the esophagus with intestinal-like glandular epithelium. BE is a consequence of chronic gastroesophageal reflux disease (GERD). Roughly 15-40% of adults in Western countries have GERD, and 8-20% of adults with GERD develop BE. Patients with BE have between a 0.12% and 1.6% annual rate of progression to EAC, and an 11.3-40-fold increase in their lifetime risk of developing EAC in comparison to the non-BE population. Currently, the guidelines for BE surveillance include periodic endoscopic examination. However, the cost-efficiency of this standard has been questioned since the incidence of EAC is low. In addition, the histologic diagnosis of dysplasia is subjective because of sampling bias and high interobserver variability. Thus, there is an urgent need to identify biomarkers associated with a high risk of progressing to EAC.

DNA methylation appears to occur early in the cancer development pathway, and thus may provide a more appropriate marker for both predicting cancer development and potentially arresting tumorigenesis. The role of epigenetic change in the pathogenesis of BE and esophageal cancer has been studied. Several methylation markers have been identified that have been suggested to discriminate between high-risk and low-risk BE including APC/p16, MGMT, PKP-1, TIMP3/TERT, RUNX3/HPP1, and OR3A4. Based on methylation and genomic change, BE and EAC samples were re-categorized into different subtypes. Dilworth et al. identified 44 methylation markers that may be able to discriminate between nondysplastic Barrett esophagus that either progress to adenocarcinoma or remain static. Hypomethylation of tumor suppressor OR3A4 (probe cg09890332) was validated in a separate cohort of samples. For OR3A4, median methylation was 67.8% in progressors versus 96.7% in non-progressors. Using receiver-operator curve (ROC) modeling, the area under curve (AUC) of this model was 0.70, and adjustment for a prevalence of 0.7% using a threshold of 58% demonstrated a sensitivity of 33.3%, and specificity of 78.6%. However, the sensitivity of this model is far too low for the clinical prediction of patients with a high-risk of progressing to EAC. Thus, there remains a need to identify useful methylation biomarkers for predicting which BE patients will progress to EAC.

### SUMMARY

Described herein are hypermethylated gene loci associated with a high risk of progressing from Barrett's esophagus (BE) to esophageal adenocarcinoma (EAC). Detecting the presence of the hypermethylated gene loci in a subject with BE enables a diagnostic strategy of closely monitoring high risk patients and can be used to guide treatment decisions. Similarly, the absence of the hypermethylated gene loci in a subject with BE can be used to determine the optimal frequency of monitoring for EAC.

Provided herein is a method of identifying a subject with BE as having a high risk of progressing to EAC. In some embodiments, the method includes measuring a methylation level of at least one gene locus of DNA in a biological sample from the subject, wherein the gene is selected from the group consisting of TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1; comparing the methylation level of the at least one gene locus to a control; and identifying the subject as having a high risk of progressing to EAC if the methylation level of the at least one gene locus is increased compared to the control. In some examples, the method includes measuring the methylation level of at least one gene locus from each of the TMEM178, KLHL14 and CPXM1 genes, and optionally further measuring methylation of at least one locus from at least one, at least two, at least, three, at least four, or at least five additional genes selected from USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1 (such as USP44, such at least one of (such as all of) TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1, such as SNCB, BMP3, CTNNA2 and NCAM1, such as SHISA3, BDNF, COL2A1, CBS, CLEC4GP1, LRAT, TTYH1 and TMEM90B, or such as TRIM71, SNCB, NTN1, VASH2 and LBXCOR1).

In some embodiments, the method further includes the step of treating the subject, such as with endoscopic mucosal resection (EMR), endoscopic submucosal surgical dissection (ESD), minimally invasive esophageal surgery, cryoablation, or radiofrequency ablation (RFA), and/or with endoscopic monitoring about every 6 months. In other embodiments, if the subject is not identified as having a high risk of progressing to EAC, the method further includes treating the subject with endoscopic monitoring at appropriate intervals, such as about every 5 to 10 years.

Also provided is a method of detecting DNA hypermethylation. In some embodiments, the method includes measuring a methylation level of at least one gene locus of DNA in a biological sample from a subject with BE, wherein the gene is selected from the group consisting of TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1; comparing the methylation level of the at least one gene locus to a control; and detecting DNA hypermethylation if the methylation level of the at least one gene locus is increased compared to the control. In some examples, the method includes measuring the methylation level of at least one gene locus from each of the TMEM178, KLHL14 and CPXM1 genes, and optionally further measuring methylation of at least one locus from at least one, at least two, at least, three, at least four, or at least five additional genes selected from USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1 (such as USP44, such at least one of (such as all of) TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1, such as SNCB, BMP3, CTNNA2 and NCAM1, such as SHISA3, BDNF, COL2A1, CBS, CLEC4GP1, LRAT, TTYH1 and TMEM90B, or such as TRIM71, SNCB, NTN1, VASH2 and LBXCOR1).

Further provided are kits for identifying a subject with BE as having a high risk of progressing to EAC and/or for detecting DNA hypermethylation of a subject with BE. In some embodiments, the kits include primers that can be used to detect DNA hypermethylation of one or more loci of the genes TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1. In some examples, the kit includes primers that amplify a nucleic acid molecule having at least one loci of each of the TMEM178, KLHL14 and CPXM1 genes, and optionally further includes primers that amplify a nucleic acid molecule comprising a locus of one or more genes selected from the group consisting of USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1 (such as USP44, such at least one of (such as all of) TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1, such as SNCB, BMP3, CTNNA2 and NCAM1, such as SHISA3, BDNF, COL2A1, CBS, CLEC4GP1, LRAT, TTYH1 and TMEM90B, or such as TRIM71, SNCB, NTN1, VASH2 and LBXCOR1).

The foregoing and other objects and features of the disclosure will become more apparent from the following detailed description, which proceeds with reference to the accompanying figures.

### BRIEF DESCRIPTION OF THE DRAWINGS

FIGS. 1A-1D: Tissues sections from subjects diagnosed with Barrett's esophagus (BE) with and without progression to esophageal adenocarcinoma (EAC). (FIG. 1A) A biopsy from a subject with EAC showed a high nuclei to cytoplasm ratio, hyperchromatic nuclei, and complicated glands with back to back glands. (FIG. 1B) A biopsy from a BE patient 10 years before EAC in FIG. 1A showed goblet cell metaplasia without dysplasia. (FIG. 1C) A biopsy from a subject with BE showed goblet cell metaplasia without dysplasia. (FIG. 1D) A biopsy taken from a subject 7 years before BE in FIG. 1C showed goblet cell metaplasia without dysplasia.

FIG. 2: Heatmap representing methylation level (β value) from 2988 CpG loci in cases of both progressing and non-progressing BE groups. The methylation level was significantly different between progressing and non-progressing BE groups.

FIG. 3: Volcano Plot of probe-level methylation p value between the progressing BE group versus the non-progressing group. Hypomethylation probes and hypermethylation probes are indicated.

FIG. 4: Heatmap representing methylation level (β value) from the top 10 hypermethylation genes in cases of both progressing and non-progressing BE groups using the Partek model. The methylation level was significantly different between the progressing and non-progressing BE groups.

FIGS. 5A-5B: Tables showing the top 10 hypermethylation gene ranking to differentiate progressing and non-progressing BE groups using the Partek model. The target ID from Illumina, UCSC RefGene name, CpG loci and p value between progressing and non-progressing BE groups in each gene are presented in these tables.

FIGS. 6A-6K: Receiver operating characteristic (ROC) curves for the top 10 hypermethylated gene loci (Partek model) individually (FIGS. 6A-6J) and in combination (FIG. 6K) to differentiate the progressing BE group from the non-progressing BE group. The specificity and sensitivity are shown in parentheses (specificity, sensitivity). Area under curve (AUC) is also indicated.

### DETAILED DESCRIPTION

### I. Abbreviations

AUC area under curve

BE Barrett's esophagus

bs-DNA bisulfate DNA

DMP differentially methylated prob

EAC esophageal adenocarcinoma

ESD endoscopic submucosal surgical dissection

EMR endoscopic mucosal resection

FFPE formalin fixed paraffin embedded

GERD gastroesophageal reflux disease

GI gastrointestinal

GWAS genome-wide association study

RFA radiofrequency ablation

ROC receiver operating characteristic

TSS transcriptional start site

### II. Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. All references, including patents and patent applications and sequences associated with the disclosed accession numbers (i.e., the sequence available on Jul. 15, 2020 or July __ 2021), are incorporated by reference in their entireties.

As used herein, the singular forms “a,” “an,” and “the,” refer to both the singular as well as plural, unless the context clearly indicates otherwise. For example, the term “a gene” includes single or plural genes and can be considered equivalent to the phrase “at least one gene.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various embodiments, the following explanations of terms are provided:

Adenocarcinoma: Carcinoma derived from glandular tissue or in which the tumor cells form recognizable glandular structures. Adenocarcinomas can be classified according to the predominant pattern of cell arrangement, such as papillary, alveolar, etc., or according to a particular product of the cells, such as mucinous adenocarcinoma. Adenocarcinomas arise in several tissues, including kidney, breast, colon, cervix, esophagus, gastric, pancreas, prostate, and lung.

Barrett's esophagus (BE): An abnormal change (intestinal metaplasia) in the cells of the esophagus, typically the lower (distal) portion of the esophagus. Barrett's esophagus is the diagnosis when portions of the normal stratified squamous epithelium lining the esophagus are replaced by intestinal columnar epithelium. Barrett's esophagus is found in 5-15% of patients who seek medical care for gastroesophageal reflux disease (GERD), although a large subgroup of patients with Barrett's esophagus do not have symptoms. Barrett's esophagus is strongly associated with esophageal adenocarcinoma, and is considered to be a premalignant condition. The main cause of Barrett's esophagus is thought to be an adaptation to chronic acid exposure from gastric reflux.

Bisulfite sequencing: The use of bisulfite treatment of DNA prior to sequencing to determine the pattern of DNA methylation. Treatment of DNA with bisulfite converts cytosine residues to uracil, while leaving 5-methylcytosine residues unaffected.

BMP3 (Bone morphogenetic protein 3): A gene encoding a secreted ligand of the TGF-β superfamily of proteins. Ligands of this family bind various TGF-β receptors leading to recruitment and activation of SMAD family transcription factors that regulate gene expression. The encoded preproprotein is proteolytically processed to generate each subunit of the disulfide-linked homodimer. The BMP3 protein suppresses osteoblast differentiation, and negatively regulates bone density, by modulating TGF-β receptor availability to other ligands. Genomic, mRNA and protein sequences of BMP3 can be found under NCBI Gene ID 651 (see NC_000004.12 for genomic sequence).

Cancer: A malignant tumor characterized by abnormal or uncontrolled cell growth. Other features often associated with cancer include metastasis, interference with the normal functioning of neighboring cells, release of cytokines or other secretory products at abnormal levels, and suppression or aggravation of inflammatory or immunological response. “Metastatic disease” refers to cancer cells that have left the original tumor site and migrate to other parts of the body, for example via the bloodstream or lymph system.

CBS (cystathionine beta-synthase): A gene encoding homotetramer enzyme that catalyzes the conversion of homocysteine to cystathionine. Defects in this gene can cause cystathionine beta-synthase deficiency (CBSD), which can lead to homocystinuria. This gene is associated with multiple cancers including ovarian cancer, hepatocellular carcinoma, gastric cancer, glioma, melanoma and breast cancer. Genomic, mRNA and protein sequences of CBS can be found under NCBI Gene ID 875 (see NC_000021.9 for genomic sequence).

Chemotherapeutic agent: Any chemical or biological agent with therapeutic usefulness in the treatment of diseases characterized by abnormal cell growth. Such diseases include tumors, neoplasms, and cancer. In one embodiment, a chemotherapeutic agent is an agent of use in treating EAC. In some examples, chemotherapeutic agents include carboplatin, paclitaxel, cisplatin, vinorelbine, folinic acid, fluorouracil, or oxaliplatin, in any combination together or with other agents. In some examples, the chemotherapeutic agents include a combination of carboplatin and paclitaxel, a combination of cisplatin and vinorelbine, and a combination of folinic acid, fluorouracil, and oxaliplatin. In some examples, the chemotherapeutic agents include one or more biologics, such as anti-cancer monoclonal antibodies (e.g., anti-VEGF, such as ranibizumab or bevacizumab, and anti-EGFR such as cetuximab or panitumumab). Exemplary chemotherapeutic agents are provided in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993, all incorporated herein by reference. Combination chemotherapy is the administration of more than one agent (such as more than one chemical chemotherapeutic agent) to treat cancer. Such a combination can be administered simultaneously, contemporaneously, or with a period of time in between.

CLEC4GP1 (C-type lectin domain family 4 member G pseudogene 1): A pseudogene on human chromosome 19. Genomic and RNA sequences of CLEC4GP1 can be found under NCBI Gene ID 440508 (see NC_000019.10 for genomic sequence).

COL2A1 (collagen type II alpha 1 chain): A gene encoding the alpha-1 chain of type II collagen, a fibrillar collagen found in cartilage and the vitreous humor of the eye. Mutations in this gene are associated with achondrogenesis, chondrodysplasia, early onset familial osteoarthritis, SED congenita, Langer-Saldino achondrogenesis, Kniest dysplasia, Stickler syndrome type I, and spondyloepimetaphyseal dysplasia Strudwick type. In addition, defects in processing chondrocalcin, a calcium binding protein that is the C-propeptide of this collagen molecule, are also associated with chondrodysplasia. COL2A1 is also known as AOM, ANFH, SEDC, STL1 and COL11A3. Genomic, mRNA and protein sequences of COL2A1 can be found under NCBI Gene ID 1280 (see NC_000012.12 for genomic sequence).

Control: A reference standard. In some embodiments, the control is a healthy subject, such as a healthy subject without BE or EAC. In other embodiments, the control is a subject with a cancer, such as EAC, or a subject with BE. In some embodiments, the control is a subject with BE who does not progress to EAC. In other embodiments, the control is a subject who progresses from BE to EAC. In still other embodiments, the control is a historical control or standard reference value or range of values (e.g., a previously tested control subject with a known prognosis or outcome or group of subjects that represent baseline or normal values). A difference between a test subject and a control can be an increase or a decrease. The difference can be a qualitative difference or a quantitative difference, for example a statistically significant difference.

CPXM1 (Carboxypeptidase X, M14 family member 1): A gene encoding a member of the carboxypeptidase family of proteins. Cloning of a comparable locus in mouse indicates that the encoded protein contains a discoidin domain and a carboxypeptidase domain, but the protein appears to lack residues necessary for carboxypeptidase activity. Genomic, mRNA and protein sequences of CPXM1 can be found under NCBI Gene ID 56265 (see NC_000020.11 for genomic sequence).

CTNNA2 (Catenin alpha 2): A gene encoding α-catenin isoform alphaN-catenin, which is involved in the development of many types of cancer. Genomic, mRNA and protein sequences of CTNNA2 can be found under NCBI Gene ID 1496 (see NC_000002.12 for genomic sequence).

Detect: To determine if an agent (such as a signal, nucleotide, amino acid, nucleic acid molecule and/or nucleotide modification, such as a methylated nucleotide, mRNA, or protein) is present or absent. In some examples, detection can include further quantification.

Diagnosis: The process of identifying a disease by its signs, symptoms and results of various tests. The conclusion reached through that process is also called “a diagnosis.” Forms of testing commonly performed include blood tests, medical imaging, and biopsy.

DNA methylation: A biological process involving the addition of methyl groups to cytosine or adenine of a DNA molecule. In mammals, methylation occurs primarily on cytosine residues to form 5-methylcytosine. Methylation of DNA can occur, for example, in promoter regions of a gene or in the gene body, and results in modulation of gene activity, such as repression of gene expression. Methylation of CpG islands in promoters is typically associated with gene repression, while methylation of a gene body is generally associated with an increase in gene transcription.

Endoscopic mucosal resection (EMR): An endoscopic technique developed for removal of sessile or flat neoplasms confined to the superficial layers (mucosa and submucosa) of the gastrointestinal (GI) tract. The mucosa and submucosa are resected from the underlying muscularis propria. An endoscopic mucosal dissection (ESD) refers to an endoscopic technique developed specifically for removing larger lesions, such as from the gastrointestinal tract, wherein the mucosa and submucosa are dissected from the other layers of the gastrointestinal tract. Both EMR and EMD typically involve injection of a substance under the targeted lesion, between the submucosa and underlying muscularis propria, to act as a cushion and elevate the submucosa and overlying mucosa. With EMR, the elevated lesion is then removed with a snare, and mobilized into a small cup by suction. With ESD, the submucosa under the lesion is dissected with a specialized knife, causing separation of the submucosa and overlying mucosa. ESD enables removal of larger and potentially deeper lesions than possible with EMR with a curative intent. Both EMR and ESD are facilitated by injection of a substance into the submucosal plane of the esophagus which effectively separates the overlying mucosa from the underlying muscularis propria, and simultaneously elevates the mucosa above the adjacent esophageal mucosa. This separation of layers and elevation of affected tissue helps the surgeon isolate, grasp, and remove the tissue of interest.

Esophageal adenocarcinoma (EAC): A type of adenocarcinoma that originates from the serial progression of Barrett's esophagus to low- and high-grade dysplasia in cells that line the distal esophagus.

Gene locus: A specific, fixed position on a chromosome where a particular gene or genetic marker is located.

Hypermethylation: An increase in the amount of methylated nucleotides in a gene or gene locus relative to a control or standard. Detecting hypermethylation can include measuring methylation using a bisulfate conversion assay or any other method of detecting DNA methylation (see, e.g., Levenson et al., Expert Rev Mol Diagn, 10(4): 481-488, 2010, or Kurdyukov and Bullock, Biology 5:3, 2016, incorporated herein by reference in their entirety).

Inhibiting or treating a disease: Inhibiting the full development of a disease or condition, for example, in a subject who is at risk for a disease, such as a subject with cancer, for example, esophageal adenocarcinoma. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

KLHL14 (Kelch-like family member 14): A gene that encodes a member of the Kelch-like gene family, whose members contain a BTB/POZ domain, a BACK domain, and several Kelch domains. The encoded protein possesses six Kelch domains and localizes to the endoplasmic reticulum, where it interacts with torsin-1A. Genomic, mRNA and protein sequences of KLHL14 can be found under NCBI Gene ID 57565 (see NC_000018.10 for genomic sequence).

LBXC01: A protein coding gene that is also known as SKOR1 (SKI family transcriptional corepressor 1). Diseases associated with SKOR1 include restless leg syndrome and essential tremor (Chen et al., Parkinsonism Relat Disord. 2018; 53:118-119). Gene Ontology (GO) annotations related to this gene include transcription corepressor activity and SMAD binding. Tumor suppression activity of Ski proteins has been observed in Ski-deficient mice, which exhibit higher sensitivity to tumor formation induced with carcinogens (Shinagawa el al., Oncogene. 2001; 20(56):8100-8108). Genomic, mRNA and protein sequences of LBCXO1 can be found under NCBI Gene ID 390598 (see NC_000015.10 for genomic sequence).

LRAT (lecithin retinol acyltransferase): A gene encoding lecithin retinol acyltransferase, an enzyme located in the endoplasmic reticulum, where it catalyzes the esterification of all-trans-retinol into all-trans-retinyl ester. This reaction is an important step in vitamin A metabolism in the visual system. Mutations in this gene have been associated with early-onset severe retinal dystrophy and Leber congenital amaurosis 14 (Sénéchal et al., Am J Ophthalmol 142(4):702-704, 2006). Genomic, mRNA and protein sequences of LRAT can be found under NCBI Gene ID 9227 (see NC_000004.12 for genomic sequence).

NCAM1 (Neural cell adhesion molecule 1): A gene encoding a cell adhesion protein which is a member of the immunoglobulin superfamily. The encoded protein is involved in cell-to-cell interactions as well as cell-matrix interactions during development and differentiation. The encoded protein has been shown to be involved in development of the nervous system, and for cells involved in the expansion of T cells and dendritic cells which play an important role in immune surveillance. Alternative splicing results in multiple transcript variants. Genomic, mRNA and protein sequences of NCAM1 can be found under NCBI Gene ID 4684 (see NG 032036.1 for genomic sequence).

NTN1 (netrin 1): A gene encoding a protein in a family of laminin-related secreted proteins. The function of this gene has not yet been defined; however, netrin is thought to be involved in axon guidance and cell migration during development. Mutations and loss of expression of netrin suggest that variation in netrin may be involved in cancer development. Genomic, mRNA and protein sequences of NTN1 can be found under NCBI Gene ID 9423 (see NC_000017.11 for genomic sequence).

Primer: Short nucleic acids, for example DNA oligonucleotides 10 nucleotides or more in length, which are annealed to a complementary target nucleic acid strand (e.g., a TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 or LBXCOR1 nucleic acid molecule, such as a region that can be hypermethylated as disclosed herein) by nucleic acid hybridization to form a hybrid between the primer and the target nucleic acid strand, then extended along the target nucleic acid strand by a polymerase enzyme. Therefore, individual primers can be used for nucleic acid sequencing. In addition, primer pairs can be used for amplification of a nucleic acid sequence, e.g., by the polymerase chain reaction (PCR) or other nucleic-acid amplification methods.

Primers can include at least 10 nucleotides complementary to the nucleic acid molecule to be sequenced. In order to enhance specificity, longer primers may also be employed, such as primers having 15, 20, 30, 40, 50, 60, 70, 80, 90 or 100 consecutive nucleotides of the complementary nucleic acid molecule to be sequenced. Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

Prognosis: A prediction of the future course of a disease, such as BE or EAC. The prediction can include determining the likelihood of a subject with BE to develop EAC, or to survive a particular amount of time (e.g., determining the likelihood that a subject will survive 1, 2, 3 or 5 years), to respond to a particular therapy (e.g., chemotherapy, EMR, ESD, esophageal surgery, cryoablation, or radiofrequency ablation), or combinations thereof.

Radiofrequency ablation (RFA): A non-surgical and minimally invasive procedure that uses electrical energy (heat) to destroy cancer tissue. RFA can be used as a treatment for BE or EAC.

Sample or biological sample: A sample of biological material obtained from a subject, which can include cells, proteins, and/or nucleic acid molecules (such as DNA and/or RNA, such as mRNA). Biological samples include all clinical samples useful for detection of disease, such as cancer, in subjects. Appropriate samples include any conventional biological samples, including clinical samples obtained from a human or veterinary subject. Exemplary samples include, without limitation, cancer samples (such as from surgery, tissue biopsy, tissue sections, or autopsy), cells, cell lysates, blood smears, cytocentrifuge preparations, cytology smears, bodily fluids (e.g., blood, plasma, serum, stool/feces, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In some embodiments herein, the biological sample is an esophageal cell sample, an esophageal biopsy, an esophageal resection, or a blood sample. Esophageal cell samples can be obtained, for example, using CYTOSPONGE™, a minimally invasive esophageal cell collection device (Januszewicz et al., Clin Gastroenterol Hepatol 17(4):647-656, 2019), or ESOPHACAP™, a gelatin capsule containing a compressed sponge attached to a tether (Zhou et al., Clin Exp Gastroenterol 12:219-229, 2019).

SHISA3 (shisa family member 3): A gene encoding a single-transmembrane protein that is one of nine members of a family of transmembrane adaptors that modulate both Wnt and fibroblast growth factor (FGF) signaling by blocking the maturation and transport of their receptors to the cell surface. Members of this family contain an N-terminal cysteine-rich domain with a distinct cysteine pattern, a single transmembrane domain, and a C-terminal proline-rich, low complexity region. The encoded protein acts as a tumor suppressor by accelerating beta-catenin degradation. Genomic, mRNA and protein sequences of SHISA3 can be found under NCBI Gene ID 152573 (see NC_000004.12 for genomic sequence).

SNCB (synuclein beta): A gene encoding a member of a small family of proteins that inhibit phospholipase D2 and may function in neuronal plasticity. The encoded protein is abundant in lesions of patients with Alzheimer disease. A mutation in this gene was found in individuals with dementia with Lewy bodies. Alternative splicing results in multiple transcript variants. Genomic, mRNA and protein sequences of SNCB can be found under NCBI Gene ID 6620 (see NG_012131.1 for genomic sequence).

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In some embodiments, the subject is human. In one example, the subject analyzed with the disclosed methods has Barrett's esophagus or esophageal adenocarcinoma.

TMEM90B: A gene encoding a protein that belongs to the in transmembrane family of proteins. A similar protein in rats is thought to regulate the development of excitatory synapses. TMEM90B is also known as synapse differentiation inducing 1 (SYNDIG1). Genomic, mRNA and protein sequences of TMEM90B can be found under NCBI Gene ID 79953 (see NC_000020.11 for genomic sequence).

TMEM178 (transmembrane protein 178): A gene in the TMEM family, which encodes proteins that span biological membranes. The TMEM proteins can be down- or up-regulated in tumor issues and are described as tumor suppressors or oncogenes. Genomic, mRNA and protein sequences of TMEM178 can be found under NCBI Gene ID 130733 (see NC_000002.12 for genomic sequence).

TRIM71 (tripartite motif containing 71): A gene encoding a protein that is an E3 ubiquitin-protein ligase that binds with miRNAs and maintains the growth and upkeep of embryonic stem cells. This gene also is involved in the G1-S phase transition of the cell cycle. TRIM71 genomic, mRNA and protein sequences can be found under NCBI Gene ID 131405 (see NC_000003.12 for genomic sequence).

TTYH1 (tweet), family member 1): A gene encoding a member of the tweety family of proteins. Members of this family function as chloride anion channels. The encoded protein functions as a calcium²⁺-independent, volume-sensitive large conductance chloride(−) channel. Genomic, mRNA and protein sequences of TTYH1 can be found under NCBI Gene ID 57348 (see NC_000019.10 for genomic sequence).

USP44 (ubiquitin-specific protease-44): A gene encoding a protease that functions as a deubiquitinating enzyme. The USP44 protein is thought to help regulate the spindle assembly checkpoint by preventing early anaphase onset. This protein specifically deubiquitinates CDC20, which stabilizes the anaphase promoting complex/cyclosome. USP44 genomic, mRNA and protein sequences can be found under NCBI Gene ID: 84101 (see NG_052622.1 for genomic sequence).

VASH2 (vasohibin 2): A gene encoding a tyrosine carboxypeptidase that removes the C-terminal tyrosine residue of alpha-tubulin, thereby regulating microtubule dynamics and function (Nieuwenhuis et al., Science. 2017; 358(6369):1453-1456). Genomic, mRNA and protein sequences of VASH2 can be found under NCBI Gene ID 79805 (see NC_000001.11 for genomic sequence).

### III. Introduction

Esophageal adenocarcinoma (EAC) has a poor prognosis with a 5-year overall survival of 15-20%. In the past three decades, the incidence of EAC has increased 600-700%. Approximately 0.11% to 1.6% of patients with Barrett's esophagus (BE), the precancerous lesion for EAC, progress to EAC annually. The guidelines for BE surveillance include periodic follow-up endoscopy, which suffers from low cost-effectiveness and inconsistent patient compliance. Only 5-10% of EAC patients have undergone BE surveillance. In addition, the histologic diagnosis of dysplasia is often associated with sampling bias and high interobserver variability. In the study disclosed herein, differences in DNA methylation between BE patients with and without progression to EAC were examined. Specifically, retrospective BE cases diagnosed 3 to 10 years prior to a diagnosis of EAC were identified. To be included in this study, each patient must have undertaken consistent surveillance endoscopy every 2-5 years. DNA samples were extracted from formalin fixed paraffin embedded (FFPE) tissue, and DNA methylation profiling of a progressing group (n=17) and a non-progressing group (n=15) were performed utilizing the Illumina Methylation EPIC microarray assay for 850,000 methylation sites. DNA methylation profile data were analyzed using three different models (Partek model, standard CHAMP model and stringent CHAMP model) to identify the most distinguishing genes.

Statistical analysis identified 4259 CpG loci with differential methylation. Using the Partek model, the most distinguishing loci (genes) between progressing subjects and non-progressing subjects were KLHL14, USP44, TMEM178, TRIM71 (2 loci), CTNNA2, NCAM1, CPXM1, SNCB, and BMP3. Based on analysis of the top 10 hypermethylated gene loci, the sensitivity, specificity and accuracy for identifying BE patients at high risk for progressing to EAC were 82.35%, 100% and 91.18%, respectively. Further analysis demonstrated that the individual hypermethylated gene loci were also associated with an increased risk with high sensitivity and specificity (see FIGS. 7B-7K).

Methylation array data were further processed using a DNA methylation analysis tool called CHAMP. With this more stringent method, 49 genes were identified that significantly differentiated the progressing and non-progressing group. The top 11 genes were identified as TMEM178, SHISA3, TTYH1, TMEM90B, KLHL14, BDNF, COL2A1, CBS, CLEC4GP1, LRAT and CPXM1. The sensitivity, specificity and accuracy to identify high-risk BE patients were all close to 100%.

The data were further analyzed using a third model, a standard stringency CHAMP model. Using this model, 257 genes were identified to significantly differentiate the progressing and non-progressing group. The top 9 genes were identified as TMEM178, NTN1, VASH2, LBXCOR1, USP44, TRIM71, KLHL14, CPXM1 and SNCB. The sensitivity, specificity and accuracy to identify high-risk BE patients ranged from 96% to 99%.

In total, 20 different genes (21 different loci) were identified as having differential methylation patterns between progressing and non-progressing BE patient samples. Thus, the disclosed hypermethylated gene loci can be used individually or in combination as biomarkers for screening BE patients for a higher risk of progressing to EAC.

### IV. Overview of Several Embodiments

The present disclosure describes the identification of hypermethylated gene loci associated with a high risk of progressing from Barrett's esophagus (BE) to esophageal adenocarcinoma (EAC). Detecting the presence of the hypermethylated gene loci in a subject with BE enables a diagnostic strategy of closely monitoring high risk patients and can be used to guide treatment decisions. Additionally, the absence of the hypermethylated gene loci in a subject with BE can be used to determine the optimal frequency of monitoring for EAC.

Provided herein is a method of identifying a subject with BE as having a high risk of progressing to EAC. In some embodiments, the method includes measuring a methylation level of at least one gene locus of DNA in a biological sample from the subject, wherein the gene is selected from the group consisting of TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 or LBXCOR1; comparing the methylation level of the at least one gene locus to a control; and identifying the subject as having a high risk of progressing to EAC if the methylation level of the at least one gene locus is increased compared to the control.

Also provided is a method of detecting DNA hypermethylation. In some embodiments, the method includes measuring a methylation level of at least one gene locus of DNA in a biological sample from a subject with BE, wherein the gene is selected from the group consisting of KLHL14, USP44, TMEM178, TRIM71, CTNNA2, NCAM1, CPXM1, SNCB and BMP3; comparing the methylation level of the at least one gene locus to a control; and detecting DNA hypermethylation if the methylation level of the at least one gene locus is increased compared to the control.

In some examples, the detected/measured methylation level/amount of the at least one gene locus (such as 1, 2, 3, 4, 5, 6, 7, 8, 9, 10, 11, 12, 13, 14, 15, 16, 17, 18, 19, 20 or 21 loci of TMEM178, KLHL14, CPXM1, USP44, TRIM71 (two loci), CTNNA2, NCAM1, SNCB, BMP3, SHISA3, BDNF, COL2A1, CBS, CLEC4GP1, LRAT, TTYH1, TMEM90B, NTN1, VASH2 and/or LBXCOR1) is increased at least 20%, at least 40%, at least 50%, at least 60%, at least 75%, at least 80%, at least 90%, at least 100%, at least 200%, at least 300%, at least 400%, or at least 500% relative to the detected/measured methylation level/amount of the at least one gene locus (such as 1, 2, 3, 4, 5, 6, 7, 8, 9, 10, 11, 12, 13, 14, 15, 16, 17, 18, 19, 20 or 21 loci of TMEM178, KLHL14, CPXM1, USP44, TRIM71 (2 loci), CTNNA2, NCAM1, SNCB, BMP3, SHISA3, BDNF, COL2A1, CBS, CLEC4GP1, LRAT, TTYH1, TMEM90B, NTN1, VASH2 and/or LBXCOR1) in a control sample or value (such as an esophageal sample from a subject(s) with BE who does not progress to EAC).

In some examples, the method includes measuring the methylation level of at least 3, at least 4, at least 5, at least 6, at least 7, at least 8, at least 9 or at least 10 gene loci. In particular examples, the method includes measuring the methylation level of at least one loci from each of the following combinations of genes: (i) TMEM178, KLHL14 and CPXM1; (ii) TMEM178, KLHL14, CPXM1 and USP44; (iii) TMEM178, KLHL14, CPXM1, USP44 and TRIM71; (iv) TMEM178, KLHL14, CPXM1, USP44, TRIM71 and SNCB; (v) TTYH1, TMEM90B and KLHL14; (vi) BDNF, COL2A1, CBS and CLEC4GP1; (vii) SHISA3, TTYH1, TMEM90B, LRAT and CPXM1; (viii) TMEM178, SHISA3, TTYH1, TMEM90B, BDNF and COL2A1; (ix) SHISA3, TTYH1, TMEM90B, KLHL14, BDNF, COL2A1 and CBS; (x) TMEM178, SHISA3, TTYH1, TMEM90B, BDNF, COL2A1, CBS and CPXM1; (xi) TMEM178, VASH2 and LBXCOR1; (xii) NTN1, VASH2, USP44 and TRIM71; (xiii) NTN1, VASH2, LBXCOR1, USP44 and KLHL14; (xiv) TMEM178, NTN1, VASH2, LBXCOR1, USP44 and KLHL14; (xv) NTN1, VASH2, LBXCOR1, TRIM71, KLHL14; CPXM1 and SNCB; (xvi) TMEM178, KLHL14, CPXM1, USP44, TRIM71, SNCB, BMP3, CTNNA2 and NCAM1; (xvii) TMEM178, KLHL14, CPXM1, SHISA3, BDNF, COL2A1, CBS, CLEC4GP1, LRAT, TTYH1 and TMEM90B; or (xviii) TMEM178, KLHL14, CPXM1, USP44, TRIM71, SNCB, NTN1, VASH2 and LBXCOR1.

In specific examples, the method includes measuring a methylation level of at least three gene loci of DNA in a biological sample from a subject with BE, wherein the at least three gene loci comprise one loci from each of the TMEM178, KLHL14 and CPXM1 genes; comparing the methylation level of the at least three gene loci to a control; and detecting DNA hypermethylation if the methylation level of the at least three gene loci is increased compared to the control. In particular examples, the method further includes measuring the methylation level of a USP44 gene locus. In some instances, the method further includes measuring the methylation level of one or more additional gene loci, wherein the gene is selected from the group consisting of TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1.

In some embodiments, the method further includes obtaining the biological sample from the subject. In specific examples, the biological sample includes a blood sample, an esophageal cell sample, an esophageal resection, or an esophageal biopsy. Esophageal cell samples can be obtained for example, using an esophageal cell collection device, such as a device that is minimally invasive (such as CYTOSPONGE™ or ESOPHACAP™). Esophageal biopsies can be obtained, for example, using an endoscope.

In some embodiments, the method further includes treating the subject identified as having a high risk of progressing to EAC. In some examples, treating the subject includes endoscopic mucosal resection (EMR), endoscopic submucosal surgical dissection (ESD), minimally invasive esophageal surgery, cryoablation, radiofrequency ablation (RFA), chemotherapy, radiation therapy, or any combination thereof. In some examples, treating the subject identified as having a high risk of progressing to EAC includes endoscopic monitoring about every 3 months, 4 months, 5 months, 6 months, 7 months, 8 months, 9 months, 10 months, 11 months or 12 months.

In other embodiments, if the subject is not identified as having a high risk of progressing to EAC, the method further includes treating the subject with endoscopic monitoring about every 4 to years, or about every 5 to 10 years, such as about every 4, 5, 6, 7, 8, 9 or 10 years.

In some examples of the methods disclosed herein, the gene locus or gene loci include at least one (such as two, or all three) of the gene loci selected from chromosome 2:39893121-39893496 (TMEM178); chromosome 18:30349690-30352302 (KLH14); and chromosome 20:2780978-2781497 (CPXM1). In specific examples, the gene loci include chromosome 12:95941906-95942979 (USP44).

In particular examples, the gene loci include at least 2, at least 3, at least 4, at least 5, at least 6, at least 7, at least 8, at least 9, at least 10, at least 11, at last 12, at least 13, at least 14, at least 15, at least 16, at least 17, at least 18, at least 19 or at least 20 of the gene loci selected from chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 2:80529677-80530846 (CTNNA2); chromosome 11:112832524-112834490 (NCAM1); chromosome 5:176056520-176057494 (SNCB); chromosome 3:32858194-32860506 (TRIM71; cg19127283); chromosome 4:81951941-81952808 (BMP3); chromosome 4:42399152-42400802 (SHISA3); chromosome 11:27743472-27744564 (BDNF); chromosome 12:48397889-48398731 (COL2A1); chromosome 21:44494624-44496989 (CBS); chromosome 19:7852932-7854557 (CLEC4GP1); chromosome 4:155663809-155664315 (LRAT); chromosome 19:54927902-54928225 (TTYH1); chromosome 20:24449844-24452037 (TMEM90B); chromosome 17:8924167-8926920 (NTN1); chromosome 1:213123647-213125092 (VASH2); or chromosome 15:68115485-68122575 (LBXCOR1).

In specific examples, the gene loci include: (i) chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 2:80529677-80530846 (CTNNA2); chromosome 11:112832524-112834490 (NCAM1); chromosome 5:176056520-176057494 (SNCB); chromosome 3:32858194-32860506 (TRIM71; cg19127283); and chromosome 4:81951941-81952808 (BMP3); (ii) chromosome 2:39893121-39893496 (TMEM178); chromosome 18:30349690-30352302 (KLH14); chromosome 20:2780978-2781497 (CPXM1); chromosome 4:42399152-42400802 (SHISA3); chromosome 11:27743472-27744564 (BDNF); chromosome 12:48397889-48398731 (COL2A1); chromosome 21:44494624-44496989 (CBS); chromosome 19:7852932-7854557 (CLEC4GP1); chromosome 4:155663809-155664315 (LRAT); chromosome 19:54927902-54928225 (TTYH1); and chromosome 20:24449844-24452037 (TMEM90B); or (iii) chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 5:176056520-176057494 (SNCB); chromosome 17:8924167-8926920 (NTN1); chromosome 1:213123647-213125092 (VASH2) and chromosome 15:68115485-68122575 (LBXCOR1).

In some embodiments of the methods disclosed herein, the method further includes extracting DNA from the biological sample prior to measuring the level of methylation. DNA extraction can be performed using any method suitable for preparation of the DNA for methylation analysis. In some examples, the method further includes converting the extracted DNA to bisulfite DNA (bs-DNA).

In some embodiments of the disclosed methods, measuring the methylation level includes bisulfite sequencing, microarray, bead array, PCR combined with sequencing, pyrosequencing, methylation-specific PCR, or endonuclease digestion (see section IV).

In some embodiments of the methods, the control is a biological sample from a healthy subject who does not have BE or EAC. In other embodiments, the control is a biological sample from a subject with BE who did not progress to EAC. In some examples, the biological sample of the control is an esophageal cell sample, an esophageal resection, an esophageal biopsy, or a blood sample.

Further provided herein are kits for identifying a subject with BE as having a high risk of progressing to EAC and/or for detecting DNA hypermethylation of a subject with BE. In some embodiments, the kit includes primers that can be used to detect DNA hypermethylation of one or more loci of the genes TMEM178, KLHL14, CPXM1, USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 or LBXCOR1. In some examples, the kit detects hypermethylation of at least one gene locus selected from chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 2:80529677-80530846 (CTNNA2); chromosome 11:112832524-112834490 (NCAM1); chromosome 5:176056520-176057494 (SNCB); chromosome 3:32858194-32860506 (TRIM71; cg19127283); chromosome 4:81951941-81952808 (BMP3); chromosome 4:42399152-42400802 (SHISA3); chromosome 11:27743472-27744564 (BDNF); chromosome 12:48397889-48398731 (COL2A1); chromosome 21:44494624-44496989 (CBS); chromosome 19:7852932-7854557 (CLEC4GP1); chromosome 4:155663809-155664315 (LRAT); chromosome 19:54927902-54928225 (TTYH1); chromosome 20:24449844-24452037 (TMEM90B); chromosome 17:8924167-8926920 (NTN1); chromosome 1:213123647-213125092 (VASH2); and chromosome 15:68115485-68122575 (LBXCOR1).

In particular examples, the kit detects hypermethylation of at least 2, at least 3, at least 4, at least 5, at least 6, at least 7, at least 8, at least 9, at least 10, at least 11, at last 12, at least 13, at least 14, at least 15, at least 16, at least 17, at least 18, at least 19 or at least 20 of the gene loci selected from chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 2:80529677-80530846 (CTNNA2); chromosome 11:112832524-112834490 (NCAM1); chromosome 5:176056520-176057494 (SNCB); chromosome 3:32858194-32860506 (TRIM71; cg19127283); chromosome 4:81951941-81952808 (BMP3); chromosome 4:42399152-42400802 (SHISA3); chromosome 11:27743472-27744564 (BDNF); chromosome 12:48397889-48398731 (COL2A1); chromosome 21:44494624-44496989 (CBS); chromosome 19:7852932-7854557 (CLEC4GP1); chromosome 4:155663809-155664315 (LRAT); chromosome 19:54927902-54928225 (TTYH1); chromosome 20:24449844-24452037 (TMEM90B); chromosome 17:8924167-8926920 (NTN1); chromosome 1:213123647-213125092 (VASH2); and chromosome 15:68115485-68122575 (LBXCOR1).

In specific non-limiting examples, the kit detects hypermethylation of 10 gene loci including chromosome 18:30349690-30352302 (KLH14); chromosome 12:95941906-95942979 (USP44); chromosome 2: chr2:39893121-39893496 (TMEM178); chromosome 3:32858194-32860506 (TRIM71; cg21124497); chromosome 2:80529677-80530846 (CTNNA2); chromosome 11:112832524-112834490 (NCAM1); chromosome 20:2780978-2781497 (CPXM1); chromosome 5:176056520-176057494 (SNCB); chromosome 3:32858194-32860506 (TRIM71; cg19127283); and chromosome 4:81951941-81952808 (BMP3).

In other specific non-limiting examples, the kit detects hypermethylation of 11 gene loci including chromosome 2:39893121-39893496 (TMEM178); chromosome 18:30349690-30352302 (KLH14); chromosome 20:2780978-2781497 (CPXM1); chromosome 4:42399152-42400802 (SHISA3); chromosome 11:27743472-27744564 (BDNF); chromosome 12:48397889-48398731 (COL2A1); chromosome 21:44494624-44496989 (CBS); chromosome 19:7852932-7854557 (CLEC4GP1); chromosome 4:155663809-155664315 (LRAT); chromosome 19:54927902-54928225 (TTYH1); and chromosome 20:24449844-24452037 (TMEM90B).

In other specific non-limiting examples, the kit detects hypermethylation of 9 gene loci including chromosome 2:39893121-39893496 (TMEM178); chromosome 18:30349690-30352302 (KLH14); chromosome 20:2780978-2781497 (CPXM1); chromosome 12:95941906-95942979 (USP44); chromosome 3:32858194-32860506 (TRIM71; cg21124497); chromosome 5:176056520-176057494 (SNCB); chromosome 17:8924167-8926920 (NTN1); chromosome 1:213123647-213125092 (VASH2) and chromosome 15:68115485-68122575 (LBXCOR1).

### IV. Methods for Detection of DNA Methylation

In vertebrates, DNA methylation involves the addition of a methyl or hydroxymethyl group to the C5 position of cytosine, typically in the context of a CpG dinucleotide. A number of different techniques have been developed to detect DNA methylation at specific gene loci. Exemplary methods are described in Kurdyukov and Bullock (Biology 5:3, 2016) and U.S. Publication Nos. 2016/0201113; 2010/0017418; and 2010/0267021, each of which is herein incorporated by reference.

In some embodiments, DNA methylation is detected and/or measured using bisulfite sequencing. In this method, DNA is subjected to bisulfite conversion, which causes deamination of non-methylated cytosine into uracil, which is then read as a thymine when sequenced. However, methylated cytosine remains resistant to bisulfite conversion and will be read as a cytosine residue upon sequencing. The sequences of bisulfite treated DNA and non-treated DNA are compared to determine which cytosines were methylated.

DNA methylation can also be detected by bisulfite treatment of DNA followed by PCR. DNA treated with bisulfite can be used directly in PCR in which uracil residues (previously unmethylated cytosine) and thymidine residues are amplified as thymidine and only 5-methylcytosine residues are amplified as cytosine residues. In this method, primers are designed to amplify a region of DNA of interest, such as a fragment containing CpG islands. The PCR products are cloned and sequenced to determine methylation sites.

In other embodiments, methylation-specific PCR is performed on bisulfate-converted DNA. In this method, two pairs of primers are used—a first set that favors amplification of methylated DNA and a second set that favors amplification of unmethylated DNA. Two PCR assays are performed for each DNA sample, and relative methylation level is determined based on the difference in the Ct values of the two PCR assays.

Pyrosequencing can also be used in the disclosed methods to detect DNA methylation. Primers can either be designed or obtained as part of a kit (for example, PyroMark CpG Assay from Qiagen) and PCR products are subjected to short-read pyrosequencing. The level of DNA methylation is determined based on the signal intensities of incorporated dGTP and dATP.

In some embodiments, an endonuclease assay, which does not require bisulfate conversion, can be utilized to detect and measure DNA methylation. The enzyme HpaI is capable of digesting the sequence “CCGC” only if it is unmethylated. In contrast, the MspI endonuclease is capable of cleaving the same sequence regardless of methylation status. Thus, separate digestions of a sample with HpaI and MspI followed by electrophoretic analysis of the digested products can identify sites of DNA methylation. Commercially available kits that utilize endonuclease-based technologies include, for example, EpiTect Methyl II PCR Array System (Qiagen). See also U.S. Patent Application Publication Nos. 2016/0017418 and 2010/0267021, which are incorporated by reference.

In other embodiments, commercially available microarray or bead array kits can be used for detection and quantification of DNA methylation. Examples include MethylationEPIC Array (Illumina), Human CpG Island Microarray Kit (Agilent), GeneChip Human promoter 1.0R Array (Affymetrix), GeneChip Human Tiling 2.0R Array Set (Affymetrix), VeraCode Methylation (Illumina), and Infinium HumanMethylation450 Bead Chip Array (Illumina).

### V. Additional Embodiments

Embodiment 1. A method of detecting DNA hypermethylation, comprising:

measuring a methylation level of at least three gene loci of DNA in a biological sample from a subject with Barrett's esophagus (BE), wherein the at least three gene loci comprise one loci from each of the TMEM178, KLHL14 and CPXM1 genes;

comparing the methylation level of the at least three gene loci to a control; and

detecting DNA hypermethylation if the methylation level of the at least three gene loci is increased compared to the control.

Embodiment 2. A method of identifying a subject with Barrett's esophagus (BE) as having a high risk of progressing to esophageal adenocarcinoma (EAC), comprising:

measuring a methylation level of at least three gene loci of DNA in a biological sample from the subject, wherein the at least three gene loci comprise one loci from each of the TMEM178, KLHL14 and CPXM1 genes;

comparing the methylation level of the at least three gene loci to a control; and

identifying the subject as having a high risk of progressing to EAC if the methylation level of the at least three gene loci is increased compared to the control.

Embodiment 3. The method of embodiment 1 or 2, further comprising obtaining the biological sample from the subject.

Embodiment 4. The method of embodiment 2 or embodiment 3, further comprising treating the subject identified as having a high risk of progressing to EAC.

Embodiment 5. The method of embodiment 4, wherein treating comprises endoscopic mucosal resection (EMR), endoscopic submucosal surgical dissection (ESD), minimally invasive esophageal surgery, cryoablation, or radiofrequency ablation (RFA).

Embodiment 6. The method of embodiment 4 or embodiment 5, wherein treating comprises endoscopic monitoring about every 6 months.

Embodiment 7. The method of embodiment 2 or embodiment 3, wherein if the subject is not identified as having a high risk of progressing to EAC, the method further comprises treating the subject with endoscopic monitoring about every 5 to 10 years.

Embodiment 8. The method of any one of embodiments 1-7, wherein the gene loci comprise:

chromosome 2:39893121-39893496 (TMEM178);

chromosome 18:30349690-30352302 (KLH14); and

chromosome 20:2780978-2781497 (CPXM1).

Embodiment 9. The method of any one of embodiments 1-7, further comprising measuring the methylation level of a USP44 gene locus.

Embodiment 10. The method of embodiment 9, wherein the USP44 gene locus comprises chromosome 12:95941906-95942979.

Embodiment 11. The method of any one of embodiments 1-10, further comprising measuring the methylation level of one or more additional gene loci, wherein the gene is selected from the group consisting of TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1.

Embodiment 12. The method of embodiment 11, comprising measuring the methylation level of one locus from each of the SNCB, BMP3, CTNNA2 and NCAM1 genes and two loci from the TRIM71 gene (target IDs: cg19127283 and cg21124497).

Embodiment 13. The method of embodiment 12, wherein the gene loci comprise:

chromosome 2:39893121-39893496 (TMEM178);

chromosome 18:30349690-30352302 (KLH14);

chromosome 20:2780978-2781497 (CPXM1);

chromosome 12:95941906-95942979 (USP44);

chromosome 3:32858194-32860506 (TRIM71; cg21124497);

chromosome 2:80529677-80530846 (CTNNA2);

chromosome 11:112832524-112834490 (NCAM1);

chromosome 5:176056520-176057494 (SNCB);

chromosome 3:32858194-32860506 (TRIM71; cg19127283); and

chromosome 4:81951941-81952808 (BMP3).

Embodiment 14. The method of embodiment 11, comprising measuring the methylation level of one locus from each of the SHISA3, BDNF, COL2A1, CBS, CLEC4GP1, LRAT, TTYH1 and TMEM90B genes.

Embodiment 15. The method of embodiment 14, wherein the gene loci comprise:

chromosome 2:39893121-39893496 (TMEM178);

chromosome 18:30349690-30352302 (KLH14);

chromosome 20:2780978-2781497 (CPXM1);

chromosome 4:42399152-42400802 (SHISA3);

chromosome 11:27743472-27744564 (BDNF);

chromosome 12:48397889-48398731 (COL2A1);

chromosome 21:44494624-44496989 (CBS);

chromosome 19:7852932-7854557 (CLEC4GP1);

chromosome 4:155663809-155664315 (LRAT);

chromosome 19:54927902-54928225 (TTYH1); and

chromosome 20:24449844-24452037 (TMEM90B).

Embodiment 16. The method of embodiment 11, comprising measuring the methylation level of one locus from each of the TRIM71, SNCB, NTN1, VASH2 and LBXCOR1 genes.

Embodiment 17. The method of embodiment 16, wherein the gene loci comprise:

chromosome 2:39893121-39893496 (TMEM178);

chromosome 18:30349690-30352302 (KLH14);

chromosome 20:2780978-2781497 (CPXM1);

chromosome 12:95941906-95942979 (USP44);

chromosome 3:32858194-32860506 (TRIM71; cg21124497);

chromosome 5:176056520-176057494 (SNCB);

chromosome 17:8924167-8926920 (NTN1);

chromosome 1:213123647-213125092 (VASH2) and

chromosome 15:68115485-68122575 (LBXCOR1).

Embodiment 18. The method of any one of embodiments 1-17, wherein the biological sample is an esophageal cell sample, an esophageal biopsy, an esophageal resection, or a blood sample.

Embodiment 19. The method of any one of embodiments 1-18, further comprising extracting DNA from the biological sample prior to measuring the level of methylation.

Embodiment 20. The method of embodiment 19, further comprising converting the extracted DNA to bisulfite DNA (bs-DNA).

Embodiment 21. The method of any one of embodiments 1-20, wherein measuring the methylation level comprises bisulfate sequencing, microarray, bead array, PCR combined with sequencing, pyrosequencing, methylation-specific PCR, or endonuclease digestion.

Embodiment 22. The method of any one of embodiments 1-21, wherein the control is a biological sample from a healthy subject who does not have BE or EAC.

Embodiment 23. The method of any one of embodiments 1-21, wherein the control is a biological sample from a subject with BE who did not progress to EAC.

Embodiment 24. The method of embodiment 22 or embodiment 23, wherein the biological sample of the control is an esophageal cell sample, an esophageal biopsy, an esophageal resection, or a blood sample.

Embodiment 25. A kit for identifying a subject with Barrett's esophagus (BE) as having a high risk of progressing to esophageal adenocarcinoma (EAC), or for detecting DNA hypermethylation of a subject with BE, comprising primers that amplify a nucleic acid molecule comprising at least one loci of each of the TMEM178, KLHL14 and CPXM1 genes.

Embodiment 26. The kit of embodiment 25, wherein the gene loci comprise:

chromosome 2:39893121-39893496 (TMEM178);

chromosome 18:30349690-30352302 (KLH14);

chromosome 20:2780978-2781497 (CPXM1)

Embodiment 27. The kit of embodiment 25 or embodiment 26, further comprising primers that amplify a nucleic acid molecule comprising a locus of one or more genes selected from the group consisting of USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1.

Embodiment 28. The kit of embodiment 27, wherein the gene loci comprise:

chromosome 2:39893121-39893496 (TMEM178);

chromosome 18:30349690-30352302 (KLH14);

chromosome 20:2780978-2781497 (CPXM1);

chromosome 12:95941906-95942979 (USP44);

chromosome 3:32858194-32860506 (TRIM71; cg21124497);

chromosome 2:80529677-80530846 (CTNNA2);

chromosome 11:112832524-112834490 (NCAM1);

chromosome 5:176056520-176057494 (SNCB);

chromosome 3:32858194-32860506 (TRIM71; cg19127283);

chromosome 4:81951941-81952808 (BMP3);

chromosome 4:42399152-42400802 (SHISA3);

chromosome 11:27743472-27744564 (BDNF);

chromosome 12:48397889-48398731 (COL2A1);

chromosome 21:44494624-44496989 (CBS);

chromosome 19:7852932-7854557 (CLEC4GP1);

chromosome 4:155663809-155664315 (LRAT);

chromosome 19:54927902-54928225 (TTYH1);

chromosome 20:24449844-24452037 (TMEM90B);

chromosome 17:8924167-8926920 (NTN1);

chromosome 1:213123647-213125092 (VASH2); and

chromosome 15:68115485-68122575 (LBXCOR1).

The following examples are provided to illustrate certain particular features and/or embodiments. These examples should not be construed to limit the disclosure to the particular features or embodiments described.

### EXAMPLES

### Example 1: Materials and Methods

This example describes the materials and experimental procedures for the studies described in Example 2.

BE Tissue with and without Progressing to EAC

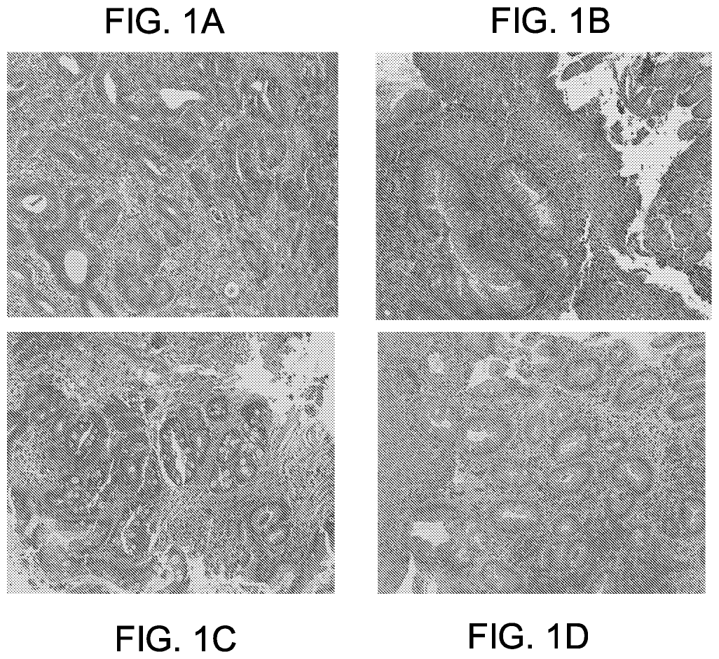

BE cases from 3-10 years prior to a diagnosis of EAC or BE were retrospectively searched from pathological software. To be included in this group, the patient must have been in a surveillance program for a minimum of 3 years and have serial biopsies over that period. Patients within the surveillance program underwent endoscopy every 2-5 years. Formalin fixed paraffin embedded (FFPE) tissues were cut in 200 μm slices and DNA was extracted. The diagnosis of each case was performed by a GI pathologist and reviewed by an additional GI pathologist for confirmation. Photos were taken from the original slides (see FIGS. 1A-1D).

### DNA Extraction

Forty non-progressing and 42 progressing FFPE BE samples were extracted for DNA using a column-based DNA extraction method (FFPE Qiagen, Hilden, Germany) following the manufacturer's instructions. All DNA samples were treated with RNase A for 1 hour at 45° C., quantified by the fluorometric method (Quant-iT PicoGreen dsDNA Assay, Life Technologies, CA, USA), and assessed for purity by NanoDrop (Thermo Scientific, MA, USA) 260/280 and 260/230 ratio measurements.

### Quality Check of FFPE DNAs

DNAs from FFPE blocks were checked for their suitability for FFPE restoration, as indicated by the Infinium HD FFPE QC Assay (Illumina Inc.), by performing a quantitative PCR with 2 ng of FFPE DNA. Delta Cq was calculated by subtracting the average value of Cq of the interrogated sample from the Cq value of a standard provided by the manufacturer. All FFPE samples in this study had a delta Cq<5, which is the recommended threshold for suitability for FFPE restoration.

### Bisulfite Conversion

Seventeen BE samples from the BE progressing group and 15 BE samples from the BE non-progressing group with over 300 ng DNA were used for methylation studies. Three hundred nanograms (300 ng) of FFPE DNA were randomly distributed on a 96-well plate and processed using the EZ-96 DNA Methylation kit (Zymo Research Corp., CA, USA) following the manufacturer's recommendations for Infinium assays. Successful conversion was verified by control PCR reactions with a primer set specific for bisulfate-converted DNA, and a primer set for unconverted DNA.

### FFPE DNA Restoration and Hybridization

Bisulfite-converted DNA (bs-DNA) from FFPE samples was processed as previously described.²² The DNA was denatured with NaOH for 10 minutes at room temperature. A 1-hour reaction at 37° C. was then performed with PPR (Primer Pre-Restore) reagent and AMR (Amplification Mix Restore reagent) reagents supplied by the kit manufacturer, in which DNA repair is accomplished. DNA was cleaned with a ZR-96 DNA Clean & Concentrator-5 kit (Zymo Research Corp.) and eluted in 13 μl of ERB. Cleaned DNA was then denatured for 2 minutes at 95° C., followed by ligation incubation at 37° C. for 1 hour with RST and CMM reagents. The resulting material was cleaned with ZR-96 DNA Clean & Concentrator-5 kit (Zymo Research Corp.) and eluted in 10 μl of distilled H₂O. Eight microliters of restored FFPE bs-DNA were then used to process through the Illumina Infinium HD Methylation Assay Protocol, as previously described.²³ Finally, 26 μl of processed sample were loaded onto MethylationEPIC BeadChips for the hybridization.

### BeadChip Processing and Data Normalization

After hybridization, the BeadChips were processed through a primer extension and an immunohistochemistry staining protocol to allow detection of a single-base extension reaction.²⁴ Finally, BeadChips were coated and then imaged on an Illumina iScan. The resulting raw data (.iDat) were background corrected and normalized by control probes using the methylation module (1.9.0) available on GenomeStudio (v2011.1) software. Methylation level of each CpG locus was calculated in GenomeStudio® Methylation module as methylation beta-value (β=intensity of the Methylated allele (M)/[intensity of the Unmethylated allele (U)+intensity of the Methylated allele (M)+100]).

### Marker Classification

CpG markers present on MethylationEPIC were classified based on their chromosome location, the Infinium chemistry used to interrogate the marker (Infinium I and II) and the feature category gene region as per UCSC annotation (TSS200, TSS1500, 5′UTR, 1^{st} Exon, Body, 3′UTR). When multiple genes or TSS were associated with a CpG site, category prioritization was applied following a 5′-prime to 3′-prime criteria (TSS200 >TSS1500 >5′UTR >1^{st} Exon >Body >3′UTR >Intergenic). Additional criteria included the location of the marker relative to the CpG island (open sea, island, shore, shelf), fantom 5-associated enhancer regions and regulatory regions described on ENCODE project such as transcription binding site sequences, open chromatin regions and digital DNase I hypersensitivity clusters.²⁵ MethylationEpic microarray includes CpG and CNG sites, CpG islands/shores/shelves/open sea, non-coding RNA (microRNAs and long non-coding RNAs) and sites surrounding the transcription start sites (−200 bp to −1,500 bp, 5′-UTRs and exons 1) for coding genes, but also for the corresponding gene bodies and 3′-UTRs, in addition to intergenic regions derived from GWAS studies.

### Identification of Differential Methylation

Before statistical analysis for differentially methylated probes (DMPs), probes with a detection p-value greater than 0.05 in more than 10% of the samples were removed (42 probes removed). Then the ANOVA test was applied to identify DMPs with a difference in the beta values between progressing and non-progressing samples, and probes with Benjamini-Hochberg (BH) adjusted p-value less than 0.05 were selected for further analysis. These analyses were carried out in Partek Genomics Suite software v6.6 (St Louis, Mo.).

### Classifier Markers for Progressed Cases

(a) Partek Genomics Suite Analysis

Model selection analysis was performed within the Partek Genomics Suite to define classifier CpG markers among the DMPs that were significant at Benjamini-Hochberg-adjusted p<0.05 and associated with a gene. This analysis was subjected to a 2-level nested cross-validation with the shrinking centroid algorithm to select variables and the nearest centroid method for classification. After the significant methylation markers were identified, the sensitivity, specificity, and accuracy were calculated. A Receiver Operating Characteristic Curve (ROC) was used to analyze area under of the curve (AUC) for the range of trade-offs between true positive (TP) and false positive (FP) error rates.

(b) CHAMP Methylation Analysis

The DNA methylation beta values were processed using a widely adopted DNA methylation analysis tool “CHAMP” for Infinium bead array data. The embedded normalization algorithm “BMIQ” was applied to the data set. An initial 865,918 probes were filtered by several criteria, including: (1) Above-background p-value <0.01; (2) not in CpG context (non-CpG probes removed); (3) known SNPs surrounding CpG region; (4) not on X and Y chromosomes; and (5) align to multiple locations in the genome.

Probes (CpG loci) with discrimination potential were defined by the following criteria: (1) Statistic FDR-q<0.05 for beta difference between two groups; (2) difference in beta value between stable and progress cases >=0.3 (by group mean value); (3) more stringent beta value cutoff was set at >=0.5 for progress cases and at <=0.2 for stable cases (common cutoff is at 0.3 for both); (4) keep only CpG loci annotated with a UCSC gene name; and (5) a most different one of multiple CpG loci for a same gene were kept in model selection procedures.

After the significant methylation markers were identified, the sensitivity, specificity, and accuracy were calculated. A ROC was used to analyze AUC for the range of trade-offs between TP and FP error rates.

### Example 2: Hypermethylation DNA Panel is a Novel Biomarker for Predicting Patients at High Risk of Progressing from Barrett's Esophagus to Esophageal Adenocarcinoma

This example describes use of the newly developed Illumina MethylationEPIC array to compare DNA methylation in BE patients who progress or do not progress to EAC in a 3- to 10-year follow-up period. This study determined that multiple DNA hypermethylation sites significantly differentiate between progressing and non-progressing BE groups.

### Patient Information

A total of 32 biopsies from FFPE blocks (17 BE cases with progression to EAC and 15 BE case without progression EAC) were passed for their suitability for FFPE restoration by the Infinium FFPE QC Assay (FIGS. 1A-1D). Demographic information of the subjects is listed in Table 1. There was no significant difference in the patients' demographic information amongst the two groups.

TABLE 1
Demographics of patients in progressing
and non-progressing BE groups

| Code | non-progressing | | progressing | |
|---|---|---|---|---|
| Age | 67.79 | | 67 | |
| Gender | M: 71.42% | F: 28.57% | M: 84.61% | F: 15.38% |
| Races | W: 100% | others: 0% | W: 91.67% | others: 8.33% |
| BMI | 30.31 | | 28.57 | |
| Smoking | 46.67% | | 63.63% | |
| Alcohol | 46.67% | | 40.00% | |

### Methylation Microarray Analysis

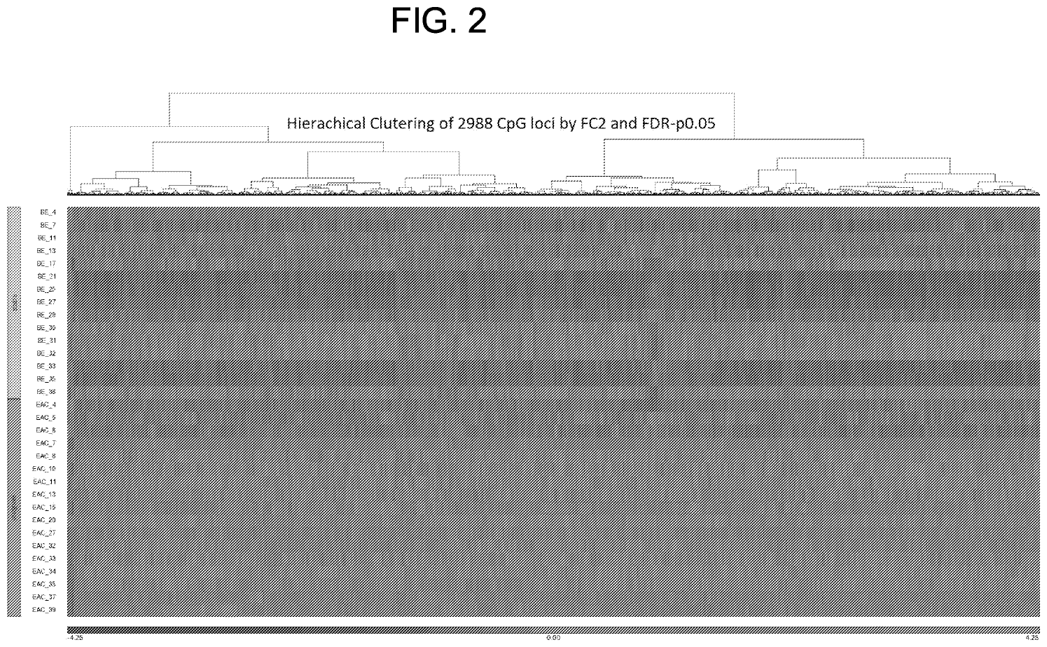

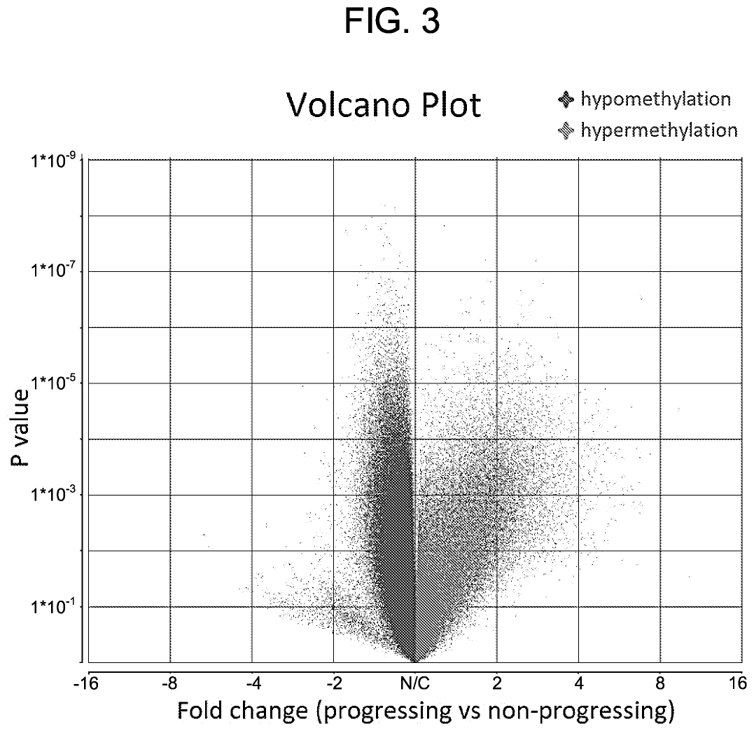

CpG methylation markers in all samples present on MethylationEPIC were classified based on their chromosome location, the Infinium chemistry and the feature category gene region. Differential methylation statistical analysis identified 4259 CpG loci with differential methylation by fold-change of progress/stale >=2 and FDR-adjusted p<0.05 between progressing and non-progressing BE groups (see heatmap, FIG. 2; and volcano plot, FIG. 3). Of these loci, 1893 genes were identified after filtering off those without annotated genes and those with missing methylation values (AVG_β) in some samples.

### Top 10 Rank of DNA Methylation by Partek Model

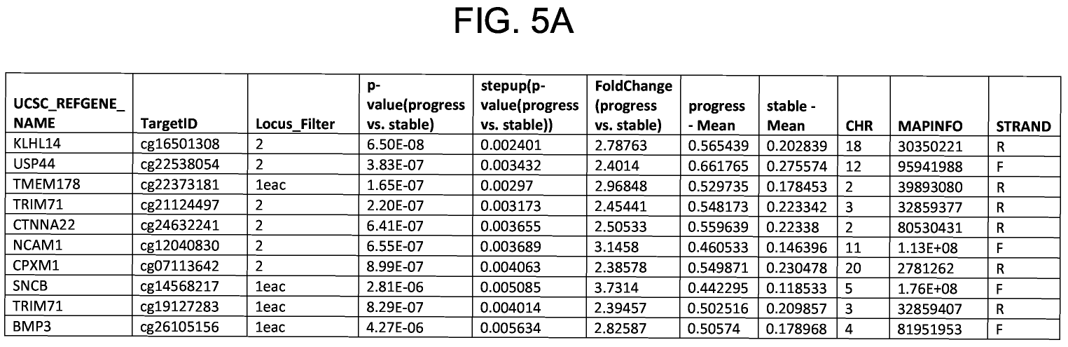

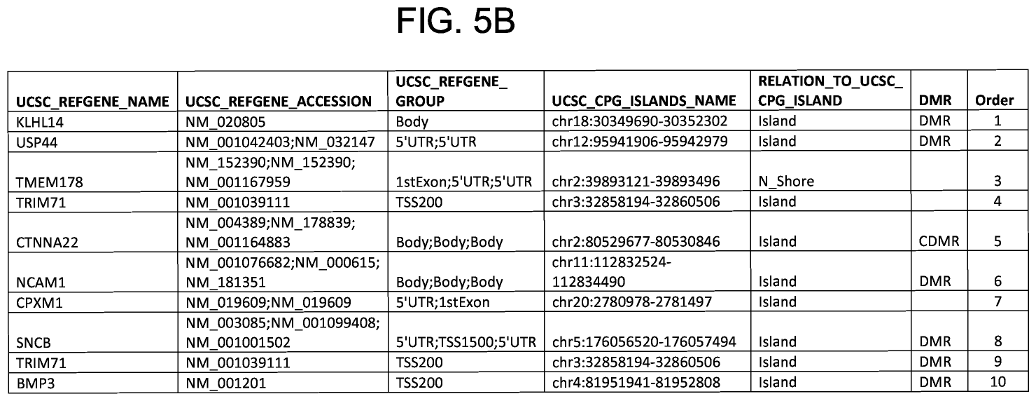

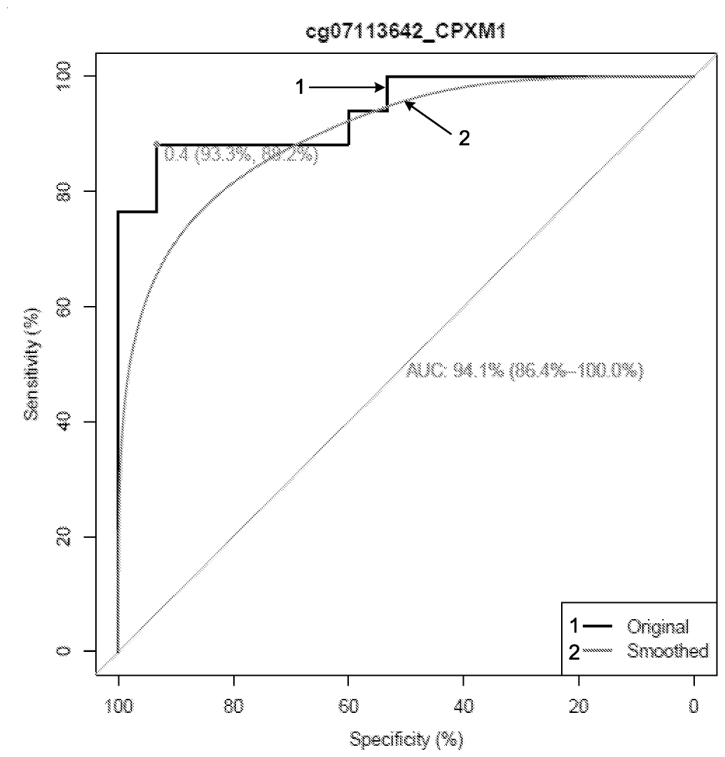

With a Partek model selection algorithms, the most distinguishing loci were picked from 1893 genes between the progressing and non-progressing samples. The top 10 hypermethylation gene biomarkers included KLHL14, USP44, TMEM178, TRIM71, CTNNA2-LRRTM1-CTNNA2, NCAM1, CPXM1, SNCB-EIF4E1B-SNCB, TRIM71, and BMP3 (FIGS. 5A-5B). Although many DNA hypomethylation genes were significantly different between the two groups, the DNA hypermethylation in progressing BE group showed significantly different from non-progressing BE group.

Analysis of Top 10 Hypermethylation Gene to Predict BE Patients with High Risk of Progressing to EAC

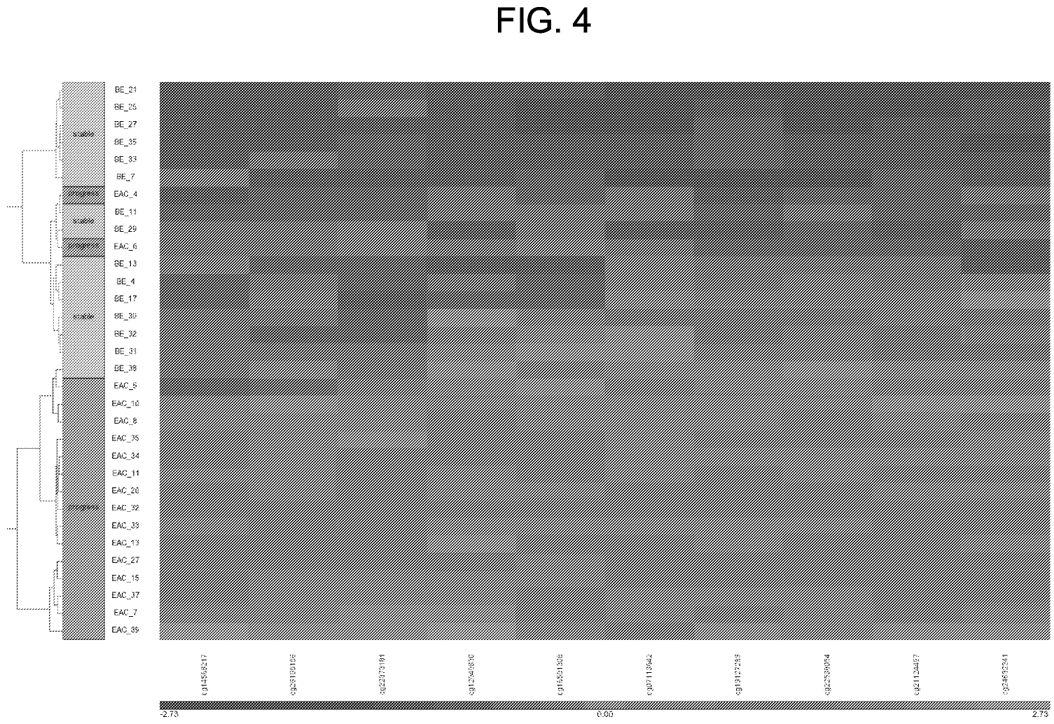

Of 17 progressing cases, 14 cases showed hypermethylation in these 10 genes: KLHL14, USP44, TMEM178, TRIM71, CTNNA2-LRRTM1-CTNNA2, NCAM1, CPXM1, SNCB-EIF4E1B-SNCB, TRIM71, and BMP3 (Table 2 and FIG. 4). Of the 15 non-progressing cases, none showed hypermethylation in these top 10 genes. The sensitivity, specificity and accuracy of top 10 gene hypermethylation to identify high risk patients to progress to EAC were 82.35%, 100% and 91.18%, respectively. Three BE progressing cases did not have these 10 gene hypermethylation. Negative predictive value and positive predictive value were 83.33% and 100%, respectively. With a receiver operating characteristics (ROC) curve analysis, the area under curve is 0.9118 without the adjustment of the incidence of EAC and 0.81 with the adjustment of the incidence of EAC.

TABLE 2
Analysis of the top 10 hypermethylation genes to predict BE patients
with a high-risk of progressing to esophageal adenocarcinoma

| Hypermethylation | BE-P group | BE-NP group | Total |
|---|---|---|---|
| positive | 14 | 0 | 14 |
| Negative | 3 | 15 | 18 |
| Total | 17 | 15 | 32 |

| Sensitivity | Specificity | PPV | NPV |
|---|---|---|---|
| 82.35% | 100% | 100% | 83.33% |
| Accuracy | AUC (ROC curve) | | |
| 91.18% | 0.9118 | | |

### Receiver Operating Characteristic (ROC) Curves

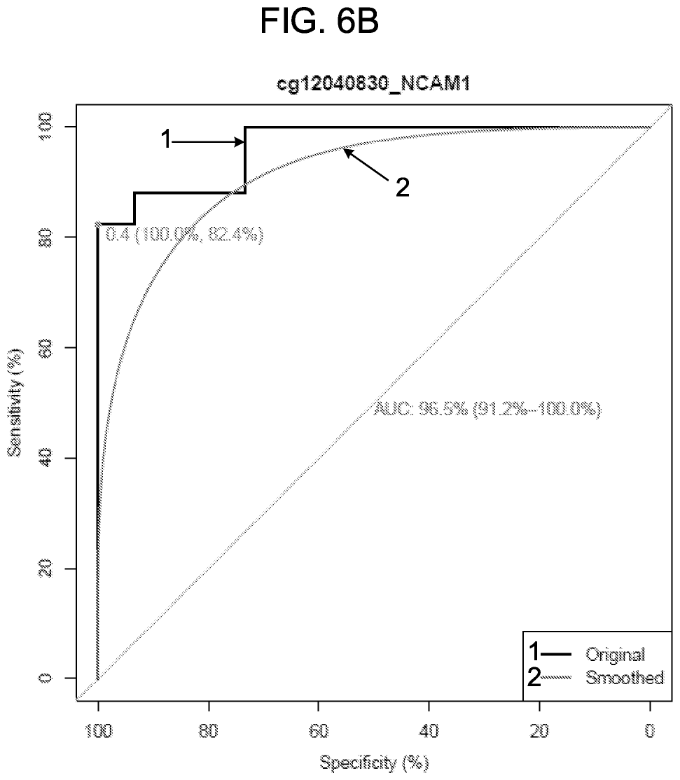

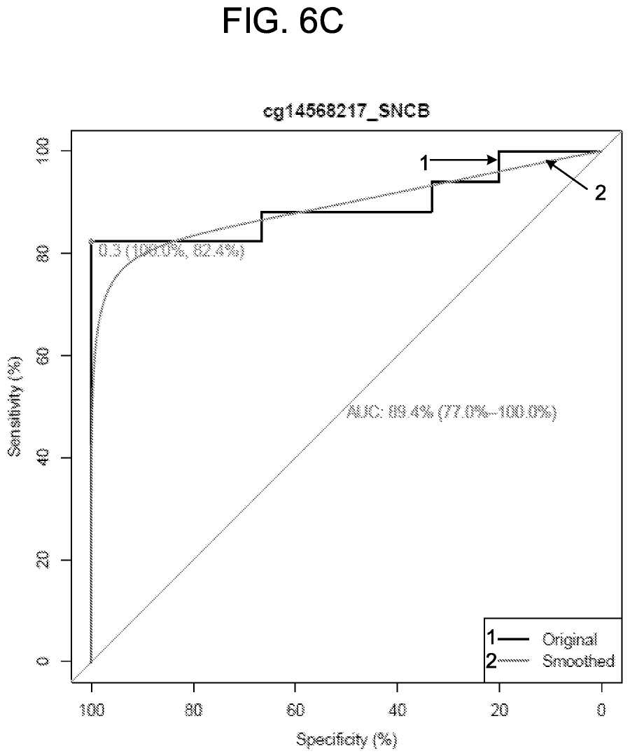

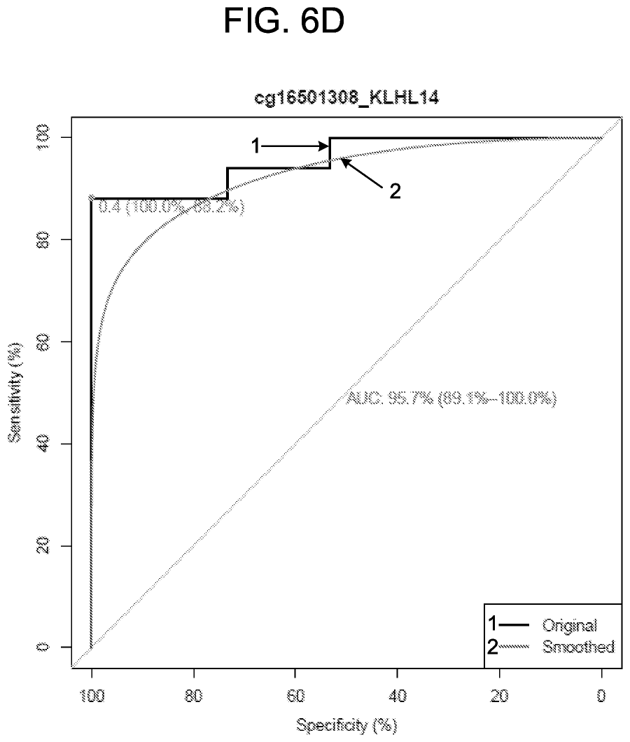

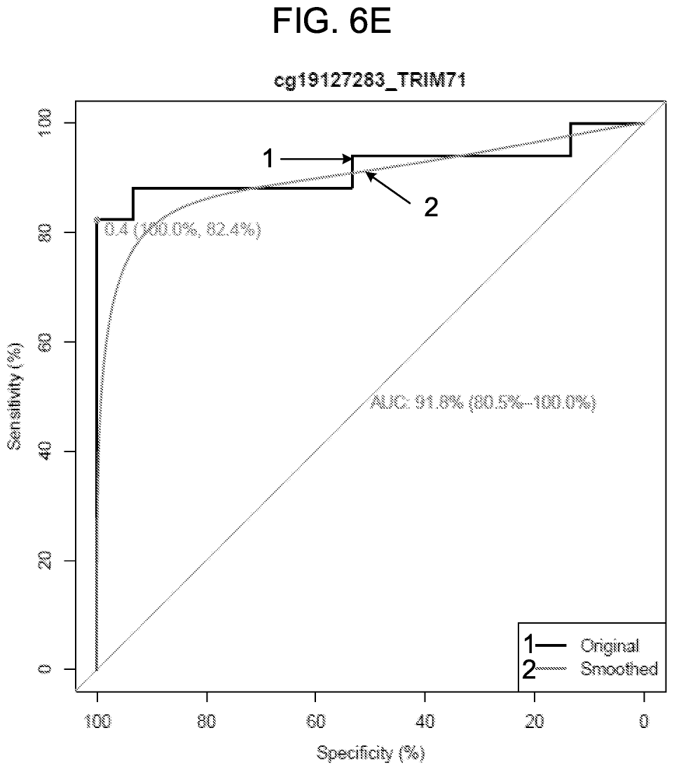

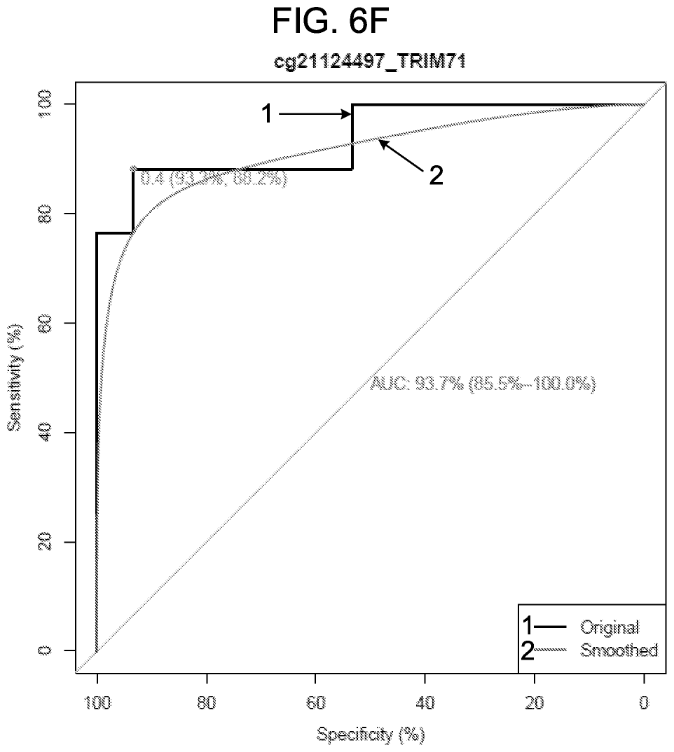

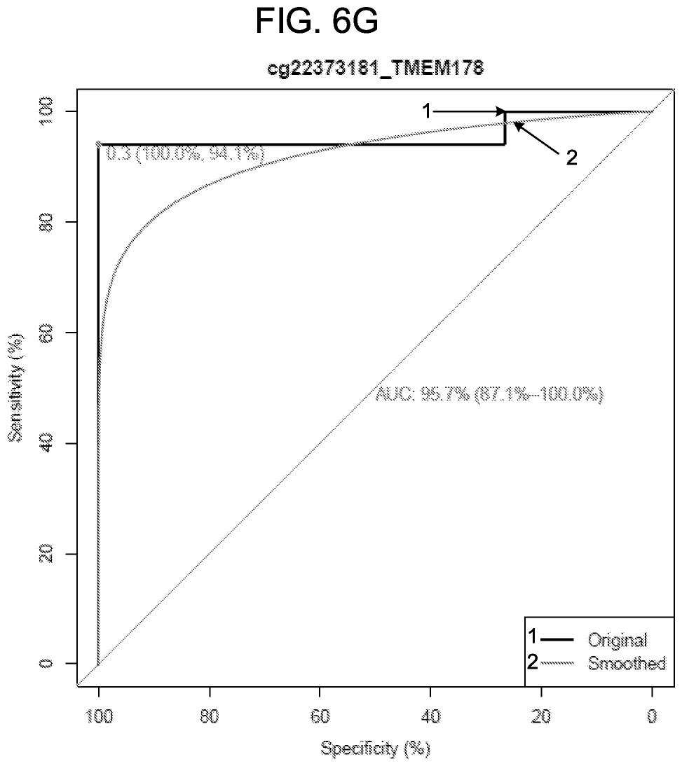

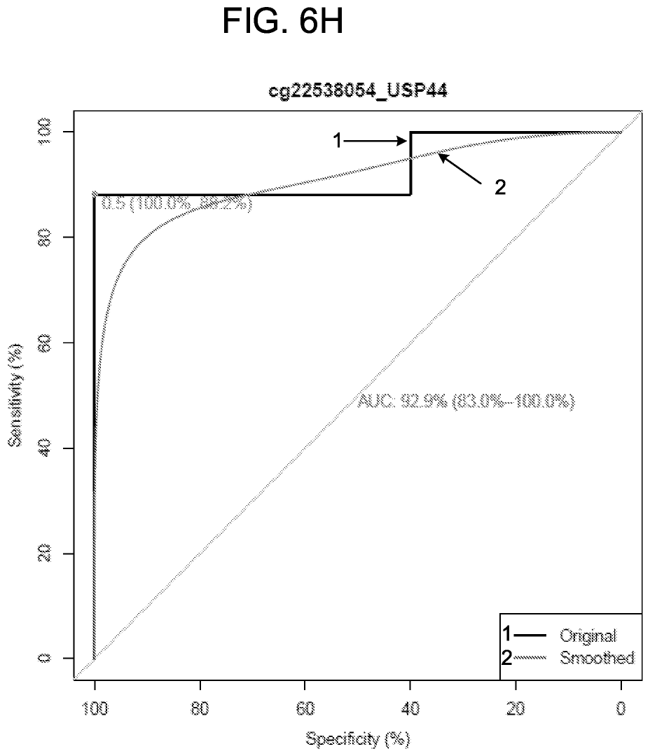

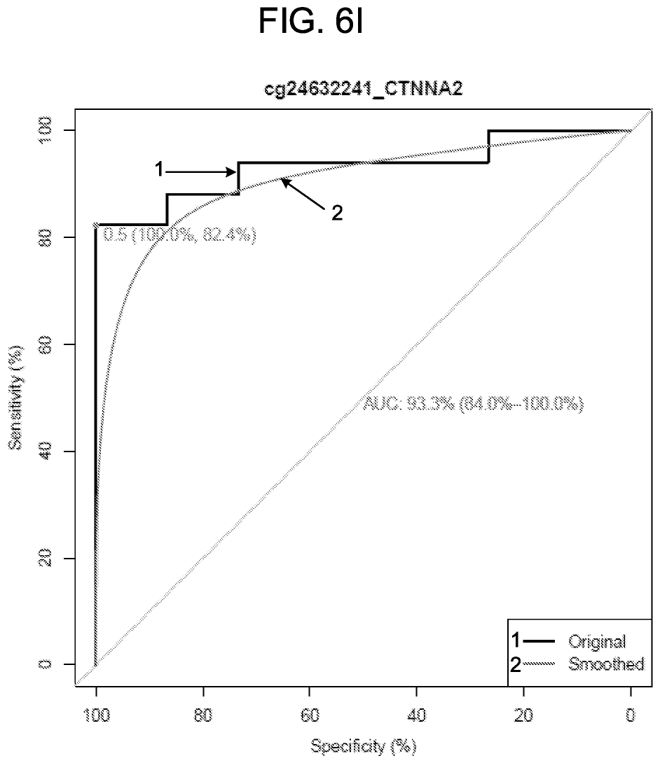

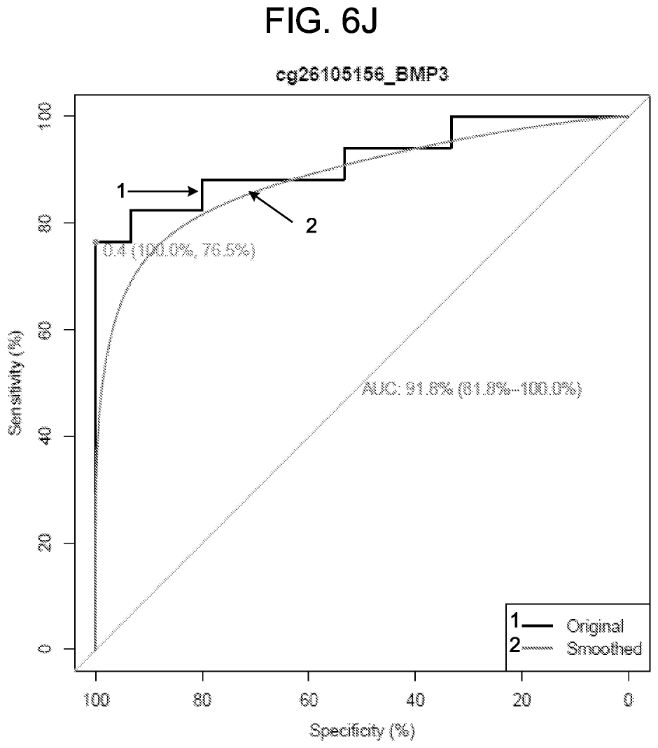

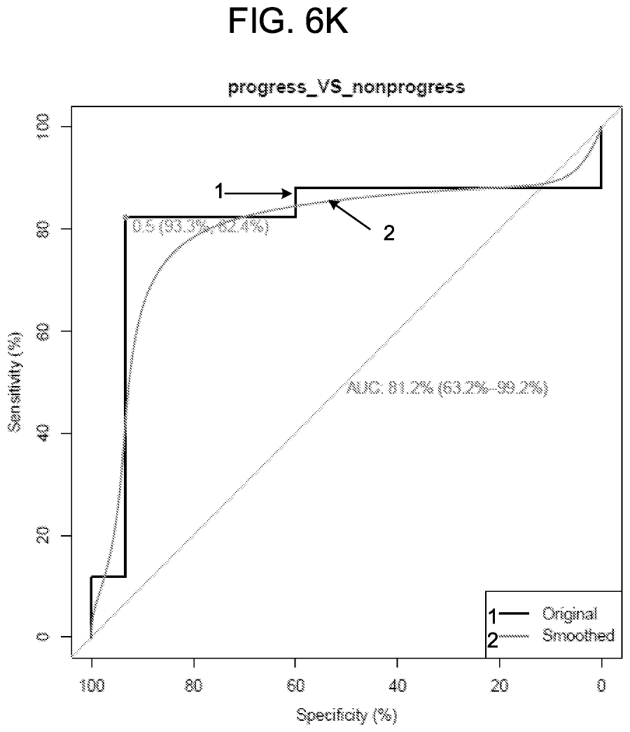

ROC curves were generated for the top 10 hypermethylated gene loci to differentiate the BE progressing group from the BE non-progressing group. FIG. 6K shows the ROC curve for all 10 gene loci in combination, while FIGS. 6A-6J show ROC curves for each gene locus individually as follows: CPXM1 (FIG. 6A), NCAM1 (FIG. 6B), SNCB (FIG. 6C), KLHL14 (FIG. 6D), TRIM71 (FIG. 6E), TRIM71 (FIG. 6F), TMEM178 (FIG. 6G), USP44 (FIG. 6H), CTNNA2 (FIG. 6I), and BMP3 (FIG. 6J). The results demonstrate that the combination of biomarkers as well as each biomarker individually, provide a high degree of sensitivity and specificity in determining whether a BE patient will progress to EAC.

Top Panels from New CHAMP Analysis Method

The Infinium methylation array data were further processed using a widely adopted DNA methylation analysis tool called “CHAMP.” With this more stringent method (β value: unmethylated <=0.2, methylated >=0.5), 49 genes were identified that significantly differentiate the progressing and non-progressing group. Based on their power to separate the two groups, each gene was ranked. The top 11 genes were identified as MEAD 78, SHISA3, TTYH1, TMEM90B KLHL14, BDNF, COL2A1, CBS, CLEC4GP1, LRAT, and CPXM1. Using a random arrangement of 11 genes, the 8 best gene panels were calculated (Table 3). The sensitivity, specificity and accuracy to identify the high-risk Barrett's esophagus patients were all close to 100%.

TABLE 3
Top 11 hypermethylation gene ranking to differentiate progressing
and non-progressing BE groups using the stringent CHAMP model

| | Model | | | | | | | |
|---|---|---|---|---|---|---|---|---|
| | 1 gene | 2 gene | 3 gene | 4 gene | 5 gene | 6 gene | 7 gene | 8 gene |
| | model | model | model | model | model | model | model | model |
| | TMEM178 | | | | | TMEM178 | | TMEM178 |
| | | SHSA3 | | | SHISA3 | SHSA3 | SHSA3 | SHSA3 |
| | | TTYH1 | TTYH1 | | TTYH1 | TTYH1 | TTYH1 | TTYH1 |
| | | | TMEM90B | | TMEM90B | TMEM90B | TMEM90B | TMEM90B |
| | | | KLHL14 | | | | KLHL14 | |
| | | | | BDNF | | BDNF | BDNF | BDNF |
| | | | | COL2A1 | | COL2A1 | COL2A1 | COL2A1 |
| | | | | CBS | | | CBS | CBS |
| | | | | CLEC4GP1 | | | | |
| | | | | | LRAT | | | |
| | | | | | CPXM1 | | | CPXM1 |
| AUC | 1.00000 | 1.00000 | 0.99996 | 0.99960 | 1.00000 | 0.99982 | 0.99982 | 1.00000 |
| Sensitivity | 1.00000 | 1.00000 | 0.99330 | 1.00000 | 0.99667 | 0.99933 | 0.99867 | 0.99067 |
| Specificity | 0.99667 | 1.00000 | 1.00000 | 0.99933 | 1.00000 | 1.00000 | 1.00000 | 1.00000 |
| Accuracy | 0.99833 | 1.00000 | 0.99967 | 0.99967 | 0.99833 | 0.99967 | 0.99933 | 0.99533 |

With the standard stringency method (β value: unmethylated <0.3, methylated >=0.3), 257 genes were identified to significantly differentiate the progressing and non-progressing group. Based on their power to separate two groups, each gene was ranked. The top 9 genes were identified as TMEM178, NTN1, VASH2, LBXCOR1, USP44, TRIM71, KLHL14, CPXM1 and SNCB. Using a random arrangement of 9 genes, the 7 best gene panels were formed (Table 4). The sensitivity, specificity and accuracy to identify the high-risk Barrett's esophagus patients were from 96% to 99%.

TABLE 4
Top 9 hypermethylation gene ranking to differentiate progressing
and non-progressing BE groups using the standard CHAMP model

| | Model | | | | | | |
|---|---|---|---|---|---|---|---|
| | 1 gene | 2 gene | 3 gene | 4 gene | 5 gene | 6 gene | 7 gene |
| | model | model | model | model | model | model | model |
| | TMEM178 | TMEM178 | TMEM178 | | | TMEM178 | |
| | | NTN1 | | NTN1 | NTN1 | NTN1 | NTN1 |
| | | | VASH2 | VASH2 | VASH2 | VASH2 | VASH2 |
| | | | LBXCOR1 | | LBXCOR1 | LBXCOR1 | LBXCOR1 |
| | | | | USP44 | USP44 | USP44 | |
| | | | | TRIM71 | | | TRIM71 |
| | | | | | KLHL14 | KLHL14 | KLHL14 |
| | | | | | | | CPXM1 |
| | | | | | | | SNCB |
| AUC | 1.00000 | 1.00000 | 1.00000 | 0.99982 | 0.99991 | 0.99951 | 0.98162 |
| Sensitivity | 1.00000 | 1.00000 | 1.00000 | 0.99733 | 0.99200 | 0.98600 | 0.96267 |
| Specificity | 0.99733 | 1.00000 | 1.00000 | 1.00000 | 1.00000 | 0.99267 | 0.97467 |
| Accuracy | 0.99867 | 1.00000 | 1.00000 | 0.99867 | 0.99600 | 0.98933 | 0.96867 |

### Summary of Results

In the studies disclosed herein, the methylation change between BE progressing and BE non-progressing samples was compared. The data were analyzed using three models: Partek, stringent CHAMP and standard CHAMP. The top genes identified using each model is summarized in Table 5. Three of the genes were identified in all three models (TMEM178, KLHL14 and CPXM1).

TABLE 5
Comparison of the top genes from the Partek model
and the stringent and standard CHAMP models

| | By Stringent | By Standard |
|---|---|---|
| By Centroid | Cutoff | Cutoff |
| Top 10 genes | Top 11 genes | Top 9 genes |
| TMEM178 | TMEM178 | TMEM178 |
| KLHL14 | KLHL14 | KLHL14 |
| CPXM1 | CPXM1 | CPXM1 |
| USP44 | SHISA3 | USP44 |
| TRIM71 | BDNF | TRIM71 |
| SNCB | COL2A1 | SNCB |
| BMP3 | CBS | NTN1 |
| CTNNA2 | CLEC4GP1 | VASH2 |
| NCAM1 | LRAT | LBXCOR1 |
| TRIM71 | TTYH1 | |
| | TMEM90B | |

### Partek Model Data

The Partek model provided a panel of 10 hypermethylated genes to predict which patients have a high risk of progressing to EAC. The sensitivity, specificity and accuracy of the top 10 hypermethylated genes to identify patients at high risk patients for progressing to EAC were 82.35%, 100% and 91.18%, respectively. In addition, differential methylation statistical analysis identified 4259 CpG loci with differential methylation by fold-change of progress/stale >=2 and FDR-adjusted p<0.05 between progressing and non-progressing BE groups.

Methylation is an early epigenetic change associated with multiple tumors. The blood based SEPT9 gene methylation assay, approved by the FDA, aims to detect aberrant methylation at the promoter region of the SEPT9 gene DNA released from colorectal cancer into the peripheral blood.^{26,27} In esophageal disease, hypermethylation and hypomethylation were identified in both EAC and BE as diagnostic or prognostic biomarkers.^{11,12,21,28,29} Based on methylation and genomic change, BE and EAC samples were grouped into 4 subtypes.¹² Subtype 1 was characterized by DNA hypermethylation with a high mutation burden. Subtype 2 was characterized by a lack of methylation at specific binding sites for transcription factors; 83% of samples of this subtype were BE and 17% were EAC. Subtype 3 did not have changes in the methylation pattern, compared with control tissue, but had a gene expression pattern that indicated immune cell infiltration. The fourth subtype was characterized by DNA hypomethylation associated with structure rearrangements, copy number alterations, with preferential amplification of CCNE1.¹² Dilworth et al. also investigated the difference in methylation between patients with and without progressing to EAC with HumanMethylation 450 array.²¹ Forty-four methylation markers were identified to discriminate between 12 progressing and 12 non-progressing BE patients. With volcano plot, the progressing group had a trend towards global hypomethylation compared to the non-progressing group, which was identified in the samples evaluated herein. However, the top 20 ranking methylation genes listed in their study were completely different from the top 10 ranking methylation genes identified herein (Table 2). There are several possible reasons to explain the differences between the two studies. First, the definition of Barrett's esophagus is different between Britain and the United States.^{30,31} The diagnosis of Barrett's esophagus in the British Gastroenterology Guideline does not require intestinal metaplasia,³⁰ but in the American College of Gastroenterology, the definition of BE is “BE should be diagnosed when there is extension of salmon-colored mucosa into the tubular esophagus extending >1 cm proximal to the gastroesophageal junction (GEJ) with biopsy confirmation of IM”³¹ All patients in the study disclosed herein had intestinal metaplasia in their histological slides (FIGS. 1A-1D). Second, the methylation method is different between the two studies. The newly developed Illumina MethylationEpic array that covers 850,000 methylation sites was used in the present study, while Dilworth et al. used the old Illumina HumanMethylation 450 array that covered 450,000 methylation sides. 400,000 methylation sites in the present study are not covered by HumanMethylation 450. Third, the analysis method in the present study is different from the study of Dilworth et al. A Parteck model selection algorithm was subjected to a 2-level nested cross-validation with the shrinking centroid algorithm to select variables and the nearest centroid method for classification. The present study identified 10 top ranking hypermethylation genes that significantly differentiated progressing and non-progressing BE patients.

The top ten hypermethylation genes were present in 15 of 17 progressing BE cases and 0 of non-progressing cases. The sensitivity, specificity and accuracy of the top 10 gene hypermethylation genes to identify high risk patients who progress to EAC were 82.35%, 100% and 91.18%, respectively. Dilworth et al. used OR3A4 hypomethylation modeling at a threshold of below 89% that can predict progression to invasive carcinoma with a sensitivity of 70.8%, and specificity of 86% in a training model. However, their validation data in other patients' groups only showed 33% sensitivity and 78.6% specificity. This level of sensitivity is too low for clinical application.

Kelch Like Family Member 14 (KLH14) gene with ID cg16501308 was ranked as the number 1 hypermethylation biomarker for predicting progressing BE patients. The protein encoded by the KLH14 gene is a part of the Kelch-like gene family members (KLHLs), which encode proteins that have the bric-a-brac, tramtrack, broad complex (BTB)/poxvirus and zinc finger (POZ) domains, BACK domain and Kelch domain. To date, 42 kinds of KLHLs have been identified.³² Several KLHLs play key roles in the ubiquitination of substrates, which are responsible for many human diseases. KLH 6, 19, 20, 22, 25 and 37 were reported to be related with various cancers including lung cancer, prostate cancer, multiple myeloma, chronic lymphocytic leukemia and gastric cancer.³²KLH 6 acts as an oncogene whose upregulation is found in 43% gastric cancer tissues.³³ KLH 19 promoter hypermethylation, which causes dysregulation of the KEAP1-Nrf2 pathway, has been discovered to be associated with a few cancers.³⁴ KLH 14 hypermethylation may play a similar role in progression of BE to EAC. KLH14 gene mutation was also identified in 6% of stomach cancer samples from the TCGA study.³⁵ In addition, there are low percentages of KLH14 gene mutations in multiple tumors including lung adenocarcinoma, squamous cell carcinoma and endometrial carcinoma.³⁶⁻³⁸

Ubiquitin-specific protease-44 (USP 44) with ID cg22538054 was ranked as the number 2 hypermethylation biomarker for predicting the progression of BE patients. USP44, located at 12q22, encodes a 712-kD amino acid, which is a member of a family of deubiquitinating enzymes that have been reported to play an important role in human cancer.³⁹⁻⁴⁵ USP44 can stabilize the protein expression of protectin in the cycle of healthy cells and prevent immature mitosis. By inhibiting USP44 expression in mice, the proportion of aneuploid cells and chromosomal instability can be increased significantly, which cause the progression to malignancy. The proliferation and migration of renal cell carcinoma are inhibited by USP44 proliferation and enhanced by USP44 knockdown.⁴³ USP44 hypermethylation in BE could also suppress the USP44 expression, which may promote the progression from BE to EAC.

Transmembrane protein 178 (TMEM178) is one gene in TMEM family, which code the proteins that spans biological membranes. The TMEM proteins can be down- or up-regulated in tumor issues and are described as tumor suppressors or oncogenes.⁴⁶ TMEM178 was identified in high grade glioma as the complex fusion DHX57:TMEM178:MAP4K3 that was sequence-validated and appears to be an activating event in pHGG.⁴⁶ In addition, TMEM178 was also reported as a negative feedback loop targeting NFATc1 to regulate bone mass 47,48

Tripartite motif-containing 71 (TRIM71) ranked number 4 and number 9 with ID cg19127283 and cg19127283, respectively, belongs to the TRIM-NHL protein family, which plays a conserved role in regulating early development and differentiation. Function of the TRIM171 protein is variable as both an oncogene and a suppressor gene.^{49,50} TRIM71 overexpression is highly associated with favorable prognosis, particularly, in TP53-mutated ovarian carcinomas, which unveils the anti-tumor function of TRIM71 in ovarian cancer development and prognosis by downregulating mutant p53s.⁴⁹ TRIM71 overexpression opposed Lin28B-induced transformation in primary cells and inhibited lung tumor formation in a mouse model. Specific knockdown of TRIM71 expression increased lung cancer cell proliferation and invasion.⁵⁰ TRIM71 hypermethylation in BE may decrease TRIM71 expression, leading to an increase in the proliferation and invasion of tumor cells.

Catenin alpha genes encode a group of α-catenin isoforms including alphaE-catenin (CTNNA1), alphaN-catenin (CTNNA2) and alphaT-catenin (CTNNA3), which are involved in the development of many types of cancer.⁵¹ In esophageal cancer cells, overexpression of novel-miR-4885, which binds to the 3′ untranslated region of CTNNA2, reduced cell adhesion and promoted epithelial-mesenchymal transition.⁵² CTNNA2 and CTNNA3 are tumor suppressors frequently mutated in laryngeal carcinoma.⁵³ Hypermethylation of the CTNNA2 gene could reduce the expression of alphaN-catenin, which may block its tumor suppressor gene function and promote the progression of BE to EAC.

Neural cell adhesion molecule 1 (NCAM1) is an immunoglobulin-like neuronal surface glycoprotein that binds to a variety of other cell adhesion proteins to mediate adhesion, guidance, and differentiation in neural growth and tumor progression.⁵⁴NCAM1 dysregulation is associated with various tumors.⁵⁴NCAM1 overexpression significantly inhibited the invasiveness of ameloblastoma AM-1 cells. Highly expressed NCAM1 suppressed the migration of ameloblastoma cells.⁵⁵ The NCAM1 gene was found to be consistently downregulated in atypical teratoid/rhabdoid tumors samples when compared to normal brain tissue.⁵⁶ The hypermethylation of NCAM1 may downregulate NCAM1 in BE to promote BE progression to EAC.

Carboxypeptidase X, M14 family member 1 (CPXM1) encodes a member of the carboxypeptidase family of proteins, which is a positive regulator of adipogenesis⁵⁷ and a collagen-binding glycoprotein.⁵⁸ The hypermethylation CPXM1 with 11 other epigenetic markers significantly distinguished patients with cancer from health volunteers, and this gene may act as a tumor suppressor gene in breast cancer cells.⁵⁹ The expression levels of CPXM1 with LAPTM4B, NGFRAP1, EMP1 genes were correlated with myelodysplastic syndrome patients' survival.⁶⁰ Using familial cancer variant prioritization pipeline is referred to as FCVPPv2 in a family with a history of papillary thyroid cancer. Only one variant with the G573R amino acid substitution in CPXM1 survived in the pipeline. The presence of this variant in the ExAC database suggests it to be a rare polymorphism or a low-penetrance risk allele.⁶¹

Beta-synuclein is a member of the synuclein protein family, found primarily in brain tissue and seen mainly in presynaptic terminals. SNCB in one of 9 mRNA panels was screened after regression analyses to establish a predictive model for classifying patients into high- and low-risk groups with significantly different overall survival times, especially for stage II and IV patients.⁶² SNCG, another member of the synuclein family, was overexpressed in 38.8% of breast cancers. However, 79% of stage III/IV breast cancers were positive for SNCG expression, whereas only 15% of stage I/II breast cancers were positive for SNCG expression. This study suggests that the expression of SNCG is stage specific for breast cancer.⁶⁴

Bone morphogenetic protein 3 (BMP3) encodes a secreted ligand of the TGF-beta (transforming growth factor-beta) superfamily of proteins. The BMP3 gene was hypermethylated and its expression was downregulated in both colorectal cancer tissues and cell lines. Expressing exogenous BMP3 in HCT116 inhibited cell growth, migration, and invasion and increased the rate of apoptosis both in vitro and in vivo. However, shRNA-mediated attenuation of endogenous BMP3 KM12 reversed such inhibitory and apoptotic effects.⁶⁵ BMP3 promoter hypermethylation was highly correlated with gastric tumors. The BMP3 gene methylation rate was 71.05% and 28.58%, respectively, in MS1 positive and negative cases (P=0.031), suggesting that BMP3 genetic instability and promoter methylation are initiated during gastric carcinogenesis.^{66,67} One study found that there was a significantly higher frequency of BMP3 methylated DNA in plasma in patients with polyps versus healthy controls with a sensitivity and specificity of 40 and 94%, respectively. That study demonstrated that BMP3 DNA methylation in plasma did not have sufficient sensitivity and it should be used in combination with other biomarkers for the detection of colorectal cancer.⁶⁸ In BE, BMP3 hypermethylation could inhibit its expression and promote progression of BE to EAC.

### CHAMP Model Data

Top 9 genes were identified using a standard analysis (β value: unmethylated <0.3, methylated >=0.3) and top 11 genes were identified using a stringent analysis (βvalue: unmethylated <=0.2, methylated >=0.5). The top 11 genes from the stringent CHAMP model, the top 9 genes from the standard CHAMP model and the top 10 genes from the Partek model were compared (Table 5). Six genes including TMEM78, USP44, TRIM71, KLHL14, CPXM1 and SNCB were shared in both the standard CHAMP model and the Partek model and 3 genes including MEM 78, KLHL14, and CPXM1 were shared in the stringent and standard CHAMP models and Partek model. With the stringent CHAMP model, the lop genes showed a greater difference than the standard analysis. In addition, the sensitivity, specificity and accuracy were close to 100%. The different genes from the CHAMP models are discussed below.

A. Stringent CHAMP Model Analysis

With the stringent CHAMP model, 8 of 11 genes including SHISA3, TTYH1, TMEM90B, BNDF, COL2A1, CBS, CLEC4GP1, and LRAT are different from both the standard CHAMP and Partek models.

The SHISA3 gene encodes a single-transmembrane protein which is one of nine members of a family of transmembrane adaptors that modulate both WNT and FGF signaling by blocking the maturation and transport of their receptors to the cell surface. The SHISA3 promoter region was hypermethylated in 61% (63/103) of breast cancer samples in comparison to the 18% of their matched normal tissues. The SHISA3 gene plays a tumor suppressing role in breast cancer through epigenetic silencing (Shahzad et al., PLoS One. 2020; 15 (7):e0236192). The promoter hypermethylation of SHISA3 is also reported in nasopharyngeal carcinoma to suppress nasopharyngeal cancer cell invasion and metastasis in vitro and in vivo (Zhang et al., Cancer Res. 2019; 79(4):747-759). The methylation rate of SHISA3 was higher in the plasma of the colorectal cancer group than in that of the healthy group. The hypermethylation of SHISA3, which inactivates the gene, is a potential cause of tumorigenesis (Tang et al., Chin J Physiol. 2021; 64(1):51-56). In esophageal cancer and Barrett's Esophagus, SHISA3 gene role is unclear.

Human homologues of tweety family 1 (TTYH1) encodes a member of the tweety family of proteins and functions as a chloride anion channel. It has been identified and characterized as a Volume-Regulated Anion Channel in Cancer Cells (Bae et al., Cells. 2019; 8 (6)). Studies showed that TTYH1 can regulate Notch signaling in neural stem cells via protein-protein interaction with Rer1 (retention in endoplasmic reticulum sorting receptor 1) (Kim et al., EMBO Rep. 2018; 19 (11)). The function for carcinogenesis is unclear.

TMEM90B, also officially known as SYNDIG1, encodes a protein that belongs to the interferon-induced transmembrane family of proteins. A similar protein in rat is thought to regulate the development of excitatory synapses. Eight cancer-specific methylated loci (ADGRB1, ANKRD13, FAM123A, GLI3, PCDHG, PPP1R16B, SLIT3, and TMEM90B) were identified in colorectal cancer by a droplet digital MethyLight assay (Cho et al., Diagnostics (Basel). 2020; 11 (1)). TMEM90B showed a sensitivity >21.4% and specificity >96.7% to diagnose colorectal cancer. The TMEM90B gene's effect on carcinogenesis is unclear.

The COL2A1 gene encodes the alpha-1 chain of type II collagen, a fibrillar collagen found in cartilage and the vitreous humor of the eye. Mutations in this gene are associated with achondrogenesis, chondrodysplasia, early onset familial osteoarthritis, SED congenita, Langer-Saldino achondrogenesis, Kniest dysplasia, Stickler syndrome type I, and spondyloepimetaphyseal dysplasia Strudwick type. In addition, defects in processing chondrocalcin, a calcium binding protein that is the C-propeptide of this collagen molecule, are also associated with chondrodysplasia. There are two transcripts identified for this gene. A COL2A1 gene with insertions, deletions and rearrangements was identified in 37% of chondrosarcoma cases. The patterns of mutation were consistent with selection for variants likely to impair normal collagen biosynthesis (Tarpey et al., Nat Genet. 2013; 45(8):923-926). COL2A1 was expressed in esophageal squamous cell carcinoma associated with poor prognosis. The results of the present study suggest that LOXL4 is a potential biomarker for patients with ESCC, as well as SUV39H1 and COL2A1, and high expression levels of these genes are associated with poor prognosis in patients with ESCC (Xie et al., Biomed Res Int. 2018; 2018:3205125). No report is found for the role of COL2A1 for carcinogenesis.

The CBS (cystathionine beta-synthase) gene encodes a homotetramer enzyme to catalyze the conversion of homocysteine to cystathionine. Defects in this gene can cause cystathionine beta-synthase deficiency (CBSD), which can lead to homocystinuria. This gene is associated with multiple cancers including ovarian cancer, hepatocellular carcinoma, gastric cancer, glioma, melanoma and breast cancer (Zhu et al., Biomed Res Int. 2018; 2018:3205125). Downregulation of CBS through promoter methylation has been observed in multiple gastric cancer cell lines, four colon cancer cell lines, glioma and hepatocellular carcinoma. However, the biological consequence of CBS epigenetic silencing in gastric cancer has not been determined (Zhu et al., Biomed Res Int. 2018; 2018:3205125).

The CLEC4GP1 gene is a pseudogene, which resembles a gene but has been mutated into an inactive form over the course of evolution. It was only present in panel 4.

The lecithin retinol acyltransferase (LRAT) gene encodes the lecithin retinol acyltransferase, located in the endoplasmic reticulum, where it catalyzes the esterification of all-trans-retinol into all-trans-retinyl ester. This reaction is an important step in vitamin A metabolism in the visual system. Mutations in this gene have been associated with early-onset severe retinal dystrophy and Leber congenital amaurosis 14. However, LRAT promoter hypermethylation was reported that the higher frequency of LRAT hypermethylation in colonic polyps and early-stage colorectal cancer indicates that it may occur early in malignant progression (Senechal et al., Am J Ophthalmol. 2006; 142(4):702-704). Esophageal adenocarcinoma derived from intestinal metaplasia, which may share a similar carcinogenesis mechanism.

B. Standard CHAMP Model Analysis

With the standard CHAMP model, 3 of 9 genes including NTN1, VASH2, and LBXCOR1, were different from both the standard. CHAMP and Partek models. Below, these three genes and their relationship with cancer development is discussed.

Netrin (NTN1) is included in a family of laminin-related secreted proteins. The function of this gene has not yet been defined; however, netrin is thought to be involved in axon guidance and cell migration during development. Mutations and loss of expression of netrin suggest that variation in netrin may be involved in cancer development. NTN1 and its gene product are strongly overexpressed in human PDAC samples. Silencing of tumor cell NTN1 inhibited tumor cell invasion in vivo. NTN1 conferred apoptosis resistance to tumor and endothelial cells in vitro, induced their invasion, and provided an adhesive substrate for tumor cells (Dumartin et al., Gastroenterology. 2010; 138(4):1595-1606). NTN1 and its receptor neogenin might also act synergistically in promoting GC cells neural invasion. Inhibiting the activity of NTN1 could be a potential strategy targeting NI in GC therapy (Yin et al., J Cancer. 2019; 10(14):3197-3207). Netrin-1 and its downstream genes have up-regulated methylation in breast cancer. The relationship between methylation and survival showed the survival was worse for hypomethylation of NTN1 in kidney renal papillary cell carcinoma (KIRP) and worse for NTNG1 in kidney renal clear cell carcinoma (KIRC). This may indicate that the carcinogenic mechanism of NTN1 and NTNG1 in pan-kidney cancers is related to promoter methylation (Hao et al., Sci Rep. 2020; 10(1):5224).

Vasohibin 2 (VASH2) encodes a tyrosine carboxypeptidase that removes the C-terminal tyrosine residue of alpha-tubulin, thereby regulating microtubule dynamics and function (Nieuwenhuis et al., Science. 2017; 358(6369):1453-1456). Critical for spindle function and accurate chromosome segregation during mitosis since microtuble detyrosination regulates mitotic spindle length and positioning (Liao et al., Cell Res. 2019; 29(7):533-5). Acts as an activator of angiogenesis: expressed in infiltrating mononuclear cells in the sprouting front to promote angiogenesis (PubMed:19204325). Using the Apc^{Min/+} mouse model, which spontaneously develops multiple intestinal adenomas and early transformation into adenocarcinomas in patients with familial adenomatous polyposis, cross-breeding of mice homozygous for a deletion of Vash2 with mice heterozygous for the APC mutation resulted in animals that showed a significant decrease in the number of polyps in the small intestine by regulating tumor angiogenesis (Kitahara et al., Mol Cancer. 2014; 13:99). VASH2 also contributes to the development of endometrial cancer by promoting angiogenesis through a paracrine mode of action (Koyanagi et al., Oncol Lett. 2013; 5(3):1058-1062).

LBXCO1, also known as SKOR1 (SKI Family Transcriptional Corepressor 1) is a protein coding gene. Diseases associated with SKOR1 include restless legs syndrome and essential tremor (Chen et al., Parkinsonism Relat Disord. 2018; 53:118-119). Gene Ontology (GO) annotations related to this gene include transcription corepressor activity and SMAD binding. An important paralog of this gene is SKOR2. Tumor suppression activity of Ski proteins has been observed in Ski-deficient mice, which exhibit higher sensitivity to tumor formation induced with carcinogens (Shinagawa et al., Oncogene. 2001; 20(56):8100-81). Defective control of TGF-β signaling is responsible for cancer induction in Barret's esophagus; patients with low-grade dysplasia exhibit low Ski and SnoN protein levels in dysplastic areas, whereas these proteins are absent in patients with high-grade dysplasia/adenocarcinoma. Neither Ski nor SnoN was expressed in normal esophageal epithelium, but both were strongly expressed in BE tissue, with intense cytoplasmic positivity. Expression of these proteins decreased markedly in dysplastic areas in patients with low-grade dysplasia and was absent in those with HGD or HGD/adenocarcinoma. Ski and SnoN proteins are overexpressed in BE and may be involved in abnormal signaling elicited by transforming growth factor-beta in this epithelium, enhancing the tumorigenesis process (Villanacci et al., Hum Pathol. 2008; 39(3):403-409). LBXCO (SKOC1) may have a similar function in BE involving tumorigenesis.

In conclusion, the studies described herein provide the first disclosure of 21 hypermethylated gene loci with a strong predictive power to differentiate BE patients who will progress and who won't progress to EAC. A panel of the hypermethylated genes (either all 21 gene loci or a subset thereof, such as a panel of three, seven or eight gene loci) can be used as a biomarker to test clinical samples, including cytological samples from ESOPHACAP™, CYTOSPONGE™ or esophageal brushing, surgical samples from biopsy or resection, and plasma DNA from the blood.

### REFERENCES

1. Pohl H, Sirovich B, Welch H G. Esophageal adenocarcinoma incidence: are we reaching the peak? Cancer Epidemiol Biomarkers Prev. 2010; 19(6): 1468-1470.
2. Njei B, McCarty T R, Birk J W. Trends in esophageal cancer survival in United States adults from 1973 to 2009: A SEER database analysis. J Gastroenterol Hepatol. 2016; 31(6):1141-1146.
3. Kambhampati S, Tieu A H, Luber B, Wang H, Meltzer S J. Risk Factors for Progression of Barrett's Esophagus to High Grade Dysplasia and Esophageal Adenocarcinoma. Sci Rep. 2020; 10(1):4899.
4. Roumans C A M, van der Bogt R D, Steyerberg E W, et al. Adherence to recommendations of Barrett's esophagus surveillance guidelines: a systematic review and meta-analysis. Endoscopy. 2020; 52(1):17-28.
5. Sharma P, Shaheen N J, Katzka D, Bergman J. AGA Clinical Practice Update on Endoscopic Treatment of Barrett's Esophagus With Dysplasia and/or Early Cancer: Expert Review. Gastroenterology. 2020; 158(3):760-769.
6. Modiano N, Gerson L B. Barrett's esophagus: Incidence, etiology, pathophysiology, prevention and treatment. Ther Clin Risk Manag. 2007; 3(6):1035-1145.
7. Karimian M, Salamati M, Azami M. The relationship between metabolic syndrome and increased risk of Barrett's esophagus: an updated systematic review and meta-analysis. BMC Gastroenterol. 2020; 20(1):138.
8. Bhat S, Coleman H G, Yousef F, et al. Risk of malignant progression in Barrett's esophagus patients: results from a large population-based study. J Natl Cancer Inst. 2011; 103(13):1049-1057.
9. Jammula S, Katz-Summercorn A C, Li X, et al. Identification of Subtypes of Barrett's Esophagus and Esophageal Adenocarcinoma Based on DNA Methylation Profiles and Integration of Transcriptome and Genome Data. Gastroenterology. 2020.
10. Mahmood N, Rabbani S A. DNA Methylation Readers and Cancer: Mechanistic and Therapeutic Applications. Front Oncol. 2019; 9:489.
11. Lu L, Liu T, Gao J, et al. Aberrant methylation of microRNA-193b in human Barrett's esophagus and esophageal adenocarcinoma. Mol Med Rep. 2016; 14(1):283-288.
12. Jammula S, Katz-Summercorn A C, Li X, et al. Identification of Subtypes of Barrett's Esophagus and Esophageal Adenocarcinoma Based on DNA Methylation Profiles and Integration of Transcriptome and Genome Data. Gastroenterology. 2020; 158(6):1682-1697 e1681.
13. Wang Y, Qin X, Wu J, et al. Association of promoter methylation of RUNX3 gene with the development of esophageal cancer: a meta analysis. PLoS One. 2014; 9 (9): e107598.
14. Kaz A M, Wong C J, Varadan V, Willis J E, Chak A, Grady W M. Erratum to: Global DNA methylation patterns in Barrett's esophagus, dysplastic Barrett's, and esophageal adenocarcinoma are associated with BMI, gender, and tobacco use. Clin Epigenetics. 2017; 9:23.
15. Kaz A M, Wong C-J, Varadan V, Willis J E, Chak A, Grady W M. Global DNA methylation patterns in Barrett's esophagus, dysplastic Barrett's, and esophageal adenocarcinoma are associated with BMI, gender, and tobacco use. Clin Epigenetics. 2016; 8:111-111.
16. Saikia S, Rehman A U, Barooah P, et al. Alteration in the expression of MGMT and RUNX3 due to non-CpG promoter methylation and their correlation with different risk factors in esophageal cancer patients. Tumour Biol. 2017; 39(5):1010428317701630.
17. Wang J S, Guo M, Montgomery E A, et al. DNA promoter hypermethylation of p16 and APC predicts neoplastic progression in Barrett's esophagus. Am J Gastroenterol. 2009; 104(9):2153-2160.
18. Kuester D, El-Rifai W, Peng D, et al. Silencing of MGMT expression by promoter hypermethylation in the metaplasia-dysplasia-carcinoma sequence of Barrett's esophagus. Cancer Lett. 2009; 275(1):117-126.
19. Clement G, Braunschweig R, Pasquier N, Bosman F T, Benhattar J. Methylation of APC, TIMP3, and TERT: a new predictive marker to distinguish Barrett's oesophagus patients at risk for malignant transformation. J Pathol. 2006; 208(1):100-107.
20. Kaz A M, Luo Y, Dzieciatkowski S, et al. Aberrantly methylated PKP1 in the progression of Barrett's esophagus to esophageal adenocarcinoma. Genes Chromosomes Cancer. 2012; 51(4):384-393.
21. Dilworth M P, Nieto T, Stockton J D, et al. Whole Genome Methylation Analysis of Nondysplastic Barrett Esophagus that Progresses to Invasive Cancer. Ann Surg. 2019; 269(3):479-485.
22. Moran S, Vizoso M, Martinez-Cardus A, et al. Validation of DNA methylation profiling in formalin-fixed paraffin-embedded samples using the Infinium HumanMethylation450 Microarray. Epigenetics. 2014; 9(6):829-833.
23. Sandoval J, Heyn H, Moran S, et al. Validation of a DNA methylation microarray for 450,000 CpG sites in the human genome. Epigenetics. 2011; 6(6):692-702.
24. Gunderson K L. Whole-genome genotyping on bead arrays. Methods Mol Biol. 2009; 529:197-213.
25. Siggens L, Ekwall K. Epigenetics, chromatin and genome organization: recent advances from the ENCODE project. J Intern Med 2014; 276(3):201-214.
26. Song L, Yu H, Jia J, Li Y. A systematic review of the performance of the SEPT9 gene methylation assay in colorectal cancer screening, monitoring, diagnosis and prognosis. Cancer Biomark. 2017; 18(4):425-432.
27. Grutzmann R, Molnar B, Pilarsky C, et al. Sensitive detection of colorectal cancer in peripheral blood by septin 9 DNA methylation assay. PLoS One. 2008; 3 (11):e3759.
28. Cancer Genome Atlas Research N, Analysis Working Group: Asan U, Agency B C C, et al. Integrated genomic characterization of oesophageal carcinoma. Nature. 2017; 541(7636):169-175.
29. Li D, Zhang L, Liu Y, et al. Specific DNA methylation markers in the diagnosis and prognosis of esophageal cancer. Aging (Albany N.Y.). 2019; 11(23):11640-11658.
30. Fitzgerald R C, di Pietro M, Ragunath K, et al. British Society of Gastroenterology guidelines on the diagnosis and management of Barrett's oesophagus. Gut. 2014; 63(1):7-42.
31. Shaheen N J, Falk G W, Iyer P G, Gerson L B, American College of G. ACG Clinical Guideline: Diagnosis and Management of Barrett's Esophagus. Am J Gastroenterol. 2016; 111(1):30-50; quiz 51.
32. Shi X, Xiang S, Cao J, et al. Kelch-like proteins: Physiological functions and relationships with diseases. Pharmacol Res. 2019; 148:104404.
33. Deng J, Guo J, Ma G, et al. Prognostic value of the cancer oncogene Kelch-like 6 in gastric cancer. Br J Surg. 2017; 104(13):1847-1856.
34. Guo Y, Yu S, Zhang C, Kong A N. Epigenetic regulation of Keapl-Nrf2 signaling. Free Radic Biol Med. 2015; 88 (Pt B):337-349.
35. Cancer Genome Atlas Research N. Comprehensive molecular characterization of gastric adenocarcinoma. Nature. 2014; 513(7517):202-209.
36. Cancer Genome Atlas Research N. Comprehensive genomic characterization of squamous cell lung cancers. Nature. 2012; 489(7417):519-525.
37. Cancer Genome Atlas Research N. Comprehensive molecular profiling of lung adenocarcinoma. Nature. 2014; 511(7511):543-550.
38. Cancer Genome Atlas Research N, Kandoth C, Schultz N, et al. Integrated genomic characterization of endometrial carcinoma. Nature. 2013; 497(7447):67-73.
39. Suresh B, Ramakrishna S, Lee H J, et al. K48- and K63-linked polyubiquitination of deubiquitinating enzyme USP44. Cell Biol Int 2010; 34(8):799-808.
40. Xiang T, Jiang H S, Zhang B T, Liu G. CircFOXO3 functions as a molecular sponge for miR-143-3p to promote the progression of gastric carcinoma via upregulating USP44. Gene. 2020; 753:144798.
41. Tropel P, Jung L, Andre C, Ndandougou A, Viville S. CpG Island Methylation Correlates with the Use of Alternative Promoters for USP44 Gene Expression in Human Pluripotent Stem Cells and Testes. Stem Cells Dev. 2017; 26(15):1100-1110.
42. Zhang Y, Foreman O, Wigle D A, et al. USP44 regulates centrosome positioning to prevent aneuploidy and suppress tumorigenesis. J Clin Invest. 2012; 122(12):4362-4374.
43. Zhou J, Wang T, Qiu T, et al. Ubiquitin-specific protease-44 inhibits the proliferation and migration of cells via inhibition of JNK pathway in clear cell renal cell carcinoma. BMC Cancer. 2020; 20(1):214.
44. Zhang Y K, Tian W Z, Zhang R S, Zhang Y J, Ma H T. Ubiquitin-specific protease 44 inhibits cell growth by suppressing AKT signaling in non-small cell lung cancer. Kaohsiung J Med Sci. 2019; 35(9):535-541.
45. Patel N B, Ostilla L A, Cuervo-Pardo L, Berdnikovs S, Chiarella S E. Gene expression of TMEM178, which encodes a negative regulator of NFATc1, decreases with the progression of asthma severity. Clin Transl Allergy. 2019; 9:38.
46. Carvalho D, Mackay A, Bjerke L, et al. The prognostic role of intragenic copy number breakpoints and identification of novel fusion genes in paediatric high grade glioma. Acta Neuropathol Commun. 2014; 2:23.
47. Decker C E, Yang Z, Rimer R, et al. TMEM178 acts in a novel negative feedback loop targeting NFATc1 to regulate bone mass. Proc Natl Acad Sci USA. 2015; 112(51):15654-15659.
48. Yang Z, Yan H, Dai W, et al. TMEM178 negatively regulates store-operated calcium entry in myeloid cells via association with STIM1. J Autoimmun. 2019; 101:94-108.
49. Chen Y, Hao Q, Wang J, et al. Ubiquitin ligase TRIM71 suppresses ovarian tumorigenesis by degrading mutant p53. Cell Death Dis. 2019; 10(10):737.
50. Yin J, Kim T H, Park N, et al. TRIM71 suppresses tumorigenesis via modulation of Lin28B-let-7-HMGA2 signaling. Oncotarget. 2016; 7(48):79854-79868.
51. Vite A, Li J, Radice G L. New functions for alpha-catenins in health and disease: from cancer to heart regeneration. Cell Tissue Res. 2015; 360(3):773-783.
52. Song J, Zhang P, Liu M, et al. Novel-miR-4885 Promotes Migration and Invasion of Esophageal Cancer Cells Through Targeting CTNNA2. DNA Cell Biol. 2019; 38(2):151-161.
53. Fanjul-Fernandez M, Quesada V, Cabanillas R, et al. Cell-cell adhesion genes CTNNA2 and CTNNA3 are tumour suppressors frequently mutated in laryngeal carcinomas. Nat Commun. 2013; 4:2531.
54. Weledji E P, Assob J C. The ubiquitous neural cell adhesion molecule (N-CAM). Ann Med Surg (Load). 2014; 3(3):77-81.
55. Guan G, Niu X, Qiao X, Wang X, Liu J, Zhong M. Upregulation of Neural Cell Adhesion Molecule 1 (NCAM1) by hsa-miR-141-3p Suppresses Ameloblastoma Cell Migration. Med Sci Monit. 2020; 26:e923491.
56. Suzuki M, Patel K, Huang C-C, et al. Loss of expression of the Neural Cell Adhesion Molecule 1 (NCAM1) in atypical teratoid/rhabdoid tumors: a new diagnostic marker? Applied Cancer Research. 2017; 37(1):14.
57. Kim Y H, Barclay J L, He J, et al. Identification of carboxypeptidase X (CPX)-1 as a positive regulator of adipogenesis. FASEB J. 2016; 30(7):2528-2540.
58. Kim Y H, O'Neill H M, Whitehead J P. Carboxypeptidase X-1 (CPX-1) is a secreted collagen-binding glycoprotein. Biochem Biophys Res Commun. 2015; 468(4):894-899.
59. Uehiro N, Sato F, Pu F, et al. Circulating cell-free DNA-based epigenetic assay can detect early breast cancer. Breast Cancer Res. 2016; 18(1):129.
60. Wang Y H, Lin C C, Yao C Y, et al. A 4-gene leukemic stem cell score can independently predict the prognosis of myelodysplastic syndrome patients. Blood Adv. 2020; 4(4):644-654.
61. Kumar A, Bandapalli O R, Paramasivam N, et al. Familial Cancer Variant Prioritization Pipeline version 2 (FCVPPv2) applied to a papillary thyroid cancer family. Sci Rep. 2018; 8(1):11635.
62. Lavedan C, Leroy E, Tones R, et al. Genomic organization and expression of the human beta-synuclein gene (SNCB). Genomics. 1998; 54(1):173-175.
63. Zheng W, Yang C, Qiu L, Feng X, Sun K, Deng H. Transcriptional information underlying the generation of CSCs and the construction of a nine-mRNA signature to improve prognosis prediction in colorectal cancer. Cancer Biol Ther. 2020:1-10.
64. Wu K, Weng Z, Tao Q, et al. Stage-specific expression of breast cancer-specific gene gamma-synuclein. Cancer Epidemiol Biomarkers Prev. 2003; 12(9):920-925.
65. Wen J, Liu X, Qi Y, et al. BMP3 suppresses colon tumorigenesis via ActRIIB/SMAD2-dependent and TAK1/JNK signaling pathways. J Exp Clin Cancer Res. 2019; 38(1):428.
66. Zhao Y, Cai L L, Wang H L, et al. 1,25-Dihydroxyvitamin D3 affects gastric cancer progression by repressing BMP3 promoter methylation. Onco Targets Ther. 2019; 12:2343-2353.
67. Chen X R, Wang J W, Li X, Zhang H, Ye Z Y. Role of BMP3 in progression of gastric carcinoma in Chinese people. World J Gastroenterol. 2010; 16(11):1409-1413.
68. Rokni P, Shariatpanahi A M, Sakhinia E, Kerachian M A. BMP3 promoter hypermethylation in plasma-derived cell-free DNA in colorectal cancer patients. Genes Genomics. 2018; 40(4):423-428.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A method of detecting DNA hypermethylation, comprising:
measuring a methylation level of at least three gene loci of DNA in a biological sample from a subject with Barrett's esophagus (BE), wherein the at least three gene loci comprise one loci from each of the TMEM178, KLHL14 and CPXM1 genes;
comparing the methylation level of the at least three gene loci to a control; and
detecting DNA hypermethylation if the methylation level of the at least three gene loci is increased compared to the control.

2. A method of identifying a subject with Barrett's esophagus (BE) as having a high risk of progressing to esophageal adenocarcinoma (EAC), comprising:
measuring a methylation level of at least three gene loci of DNA in a biological sample from the subject, wherein the at least three gene loci comprise one loci from each of the TMEM178, KLHL14 and CPXM1 genes;
comparing the methylation level of the at least three gene loci to a control;
identifying the subject as having a high risk of progressing to EAC if the methylation level of the at least three gene loci is increased compared to the control; and
treating the subject identified as having a high risk of progressing to EAC, or if the subject is not identified as having a high risk of progressing to EAC, treating the subject with endoscopic monitoring about every 5 to 10 years.

3. The method of claim 2, further comprising obtaining the biological sample from the subject.

4. (canceled)

5. The method of claim 2, wherein treating the subject identified as having a high risk of progression to EAC comprises:
endoscopic mucosal resection (EMR), endoscopic submucosal surgical dissection (ESD), minimally invasive esophageal surgery, cryoablation, or radiofrequency ablation (RFA); and/or
endoscopic monitoring about every 6 months.

6-7. (canceled)

8. The method of claim 1, wherein the gene loci comprise:
chromosome 2:39893121-39893496 (TMEM178);
chromosome 18:30349690-30352302 (KLH14); and
chromosome 20:2780978-2781497 (CPXM1).

9. The method of claim 1, further comprising measuring the methylation level of a USP44 gene locus.

10. The method of claim 9, wherein the USP44 gene locus comprises chromosome 12:95941906-95942979.

11. The method of claim 1, further comprising measuring the methylation level of one or more additional gene loci, wherein the gene is selected from the group consisting of TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1.

12. The method of claim 11, comprising:
i) measuring the methylation level of one locus from each of the SNCB, BMP3, CTNNA2 and NCAM1 genes and two loci from the TRIM71 gene;
ii) measuring the methylation level of one locus from each of the SHISA3, BDNF, COL2A1, CBS, CLEC4GP1, LRAT, TTYH1 and TMEM90B genes; and/or
iii) measuring the methylation level of one locus from each of the TRIM71, SNCB, NTN1, VASH2 and LBXCOR1 genes.

13. The method of claim 12, wherein the gene loci comprise:
chromosome 2:39893121-39893496 (TMEM178);
chromosome 18:30349690-30352302 (KLH14);
chromosome 20:2780978-2781497 (CPXM1);
chromosome 12:95941906-95942979 (USP44);
chromosome 3:32858194-32860506 (TRIM71; cg21124497);
chromosome 2:80529677-80530846 (CTNNA2);
chromosome 11:112832524-112834490 (NCAM1);
chromosome 5:176056520-176057494 (SNCB);
chromosome 3:32858194-32860506 (TRIM71; cg19127283); and
chromosome 4:81951941-81952808 (BMP3).

14. (canceled)

15. The method of claim 12, wherein the gene loci comprise:
chromosome 2:39893121-39893496 (TMEM178);
chromosome 18:30349690-30352302 (KLH14);
chromosome 20:2780978-2781497 (CPXM1);
chromosome 4:42399152-42400802 (SHISA3);
chromosome 11:27743472-27744564 (BDNF);
chromosome 12:48397889-48398731 (COL2A1);
chromosome 21:44494624-44496989 (CBS);
chromosome 19:7852932-7854557 (CLEC4GP1);
chromosome 4:155663809-155664315 (LRAT);
chromosome 19:54927902-54928225 (TTYH1); and
chromosome 20:24449844-24452037 (TMEM90B).

16. (canceled)

17. The method of claim 12, wherein the gene loci comprise:
chromosome 2:39893121-39893496 (TMEM178);
chromosome 18:30349690-30352302 (KLH14);
chromosome 20:2780978-2781497 (CPXM1);
chromosome 12:95941906-95942979 (USP44);
chromosome 3:32858194-32860506 (TRIM71; cg21124497);
chromosome 5:176056520-176057494 (SNCB);
chromosome 17:8924167-8926920 (NTN1);
chromosome 1:213123647-213125092 (VASH2) and
chromosome 15:68115485-68122575 (LBXCOR1).

18. The method of claim 1, wherein the biological sample is an esophageal cell sample, an esophageal biopsy, an esophageal resection, or a blood sample.

19. The method of claim 1, further comprising extracting DNA from the biological sample prior to measuring the level of methylation and/or converting; the extracted DNA to bisulfite DNA (bs-DNA).

20. (canceled)

21. The method of claim 1, wherein measuring the methylation level comprises bisulfite sequencing, microarray, bead array, PCR combined with sequencing, pyrosequencing, methylation-specific PCR, or endonuclease digestion.

22. The method of claim 1, wherein the control is a biological sample from a healthy subject who does not have BE or EAC, or a biological sample from a subject with BE who did not progress to EAC.

23. (canceled)

24. The method of claim 22, wherein the biological sample of the control is an esophageal cell sample, an esophageal biopsy, an esophageal resection, or a blood sample.

25. A kit, comprising primers that amplify a nucleic acid molecule comprising at least one loci of each of the TMEM178, KLHL14 and CPXM1 genes.

26. The kit of claim 25, wherein the gene loci comprise:
chromosome 2:39893121-39893496 (TMEM178);
chromosome 18:30349690-30352302 (KLH14);
chromosome 20:2780978-2781497 (CPXM1)

27. The kit of claim 25, further comprising primers that amplify a nucleic acid molecule comprising a locus of one or more genes selected from the group consisting of USP44, TRIM71, CTNNA2, NCAM1, SNCB, BMP3, SHISA3, BDNF, COL2A1, CBS, CLEC4GP1, LRAT, TTYH1, TMEM90B, NTN1, VASH2 and LBXCOR1.

28. (canceled)
